(19) United States
(12) Patent Application Publication
(10) Pub. No.: US 2018/0085677 A1
(43) Pub. Date: Mar. 29, 2018

(54) MIST GENERATING DEVICE

(71) Applicant: TOMY COMPANY, LTD., Tokyo (JP)

(72) Inventors: Shima ATSUZAWA, Tokyo (JP); Hiroshi SHINOHARA, Tokyo (JP); Mamoru SUZUKI, Tokyo (JP); Saburo WATANABE, Tokyo (JP)

(73) Assignee: TOMY COMPANY, LTD., Tokyo (JP)

(21) Appl. No.: 15/715,675

(22) Filed: Sep. 26, 2017

(30) Foreign Application Priority Data
Sep. 26, 2016 (JP) 2016-187270

(51) Int. Cl. A63H 19/14; B05B 17/00

(52) U.S. Cl. CPC A63H 19/14; A61M 11/005; B05B 17/0646

(57) ABSTRACT

A mist generating device including a vibrating plate which vibrates at a high frequency and a liquid supply mechanism for supplying a conductive liquid such as water to the vibrating plate and generating a mist by bringing the liquid supplied through the liquid supply mechanism into contact with the vibrating plate for atomization, the mist generating device further including liquid-contact detecting unit for detecting presence of contact of the liquid with the vibrating plate; and protective operation performing unit for performing a protective operation for preventing idle vibration of the vibrating plate when the liquid-contact detecting unit detects non-contact of the liquid with the vibrating plate.

### CROSS REFERENCE TO RELATED APPLICATIONS

This application claims priority under 35 U.S.C. 119 from Japanese Patent Application No. 2016-187270, filed on Sep. 26, 2016, which is now Japanese Patent No. 6144398, granted on May 19, 2017, the entire contents of which are incorporated herein by reference.

### FIELD

The present disclosure relates to a mist generating device for generating a mist by bringing a liquid such as water into contact with a vibrating plate which vibrates at a high frequency for atomization.

### BACKGROUND

A mist generating device for bringing the liquid (water, for example) supplied through a liquid supply mechanism into contact with a vibrating plate which vibrates at a high frequency for atomization is widely employed in various toys performing effects of smoke (a steam locomotive toy ejecting smoke from a funnel, an automobile toy blowing out smoke from an exhaust pipe, a water fountain toy blowing up water smoke and the like) (see Japanese Patent Laid-Open No. 04-150968, Japanese Utility Model Registration Laid-Open No. 05-070592 and Japanese Patent No. 3744931, for example).

As the vibrating plate, various structures are known such as a vibrator itself constituted by sandwiching a piezoelectric material between a pair of driving electrodes (see Japanese Utility Model Registration Laid-Open No. 05-070592) or a metal tongue cantilever-supported by the aforementioned vibrator (see Japanese Patent Laid-Open No. 04-150968 and Japanese Patent No. 3744931).

As the liquid supply mechanism, too, various structures are known such as a mechanism for generating a mist by dripping a liquid stored in a liquid storage tank onto the vibrating plate in a horizontal posture through a tube with a flow regulating valve (Japanese Patent Laid-Open No. 04-150968 and Japanese Utility Model Registration Laid-Open No. 05-070592) or a mechanism for generating a mist from an upper surface side by supplying a liquid to a lower surface of the vibrating plate through a liquid retaining material such as a sponge placed on the lower surface of the vibrating plate with fine holes and in a substantially horizontal posture (Japanese Patent No. 3744931) and the like.

One of failures of this type of mist generating devices is defective generation or incapable generation of mist due to aging degradation or breakage of a vibrating plate. The inventors found that its cause is accumulation of fatigue (metal fatigue accumulation) of the vibrating plate based on high-frequency vibration in a state where a liquid is not in contact (hereinafter referred to as “idle vibration”) as the result of keen examination.

The present disclosure was made in view of the aforementioned finding and has an object to prevent defective generation or incapable generation of mist due to aging degradation or breakage of the vibrating plate in this type of mist generating devices.

### SUMMARY

It is considered that the aforementioned technical problem can be solved by a mist generating device according to the present disclosure having the following constitution.

That is, the mist generating device according to the present disclosure has a vibrating plate which vibrates at a high frequency and a liquid supply mechanism for supplying a conductive liquid such as water to the vibrating plate and generating a mist by bringing the liquid supplied through the liquid supply mechanism into contact with the vibrating plate for atomization, the mist generating device further including liquid-contact detecting unit for detecting presence of contact of the liquid with the vibrating plate; and protective operation performing unit for performing a protective operation for preventing idle vibration of the vibrating plate when the liquid-contact detecting unit detects non-contact of the liquid with the vibrating plate.

According to such constitution, when such a state emerges where the liquid which is the mist material is not brought into contact with the vibrating plate due to various reasons caused by the structure of the liquid supply mechanism such that the liquid storage tank is emptied, a liquid supply path from the liquid storage tank to the vibrating plate is clogged, a sponge which is a liquid retaining material is dried or the like, the protective operation for preventing idle vibration of the vibrating plate is immediately performed and as a result, defective generation or incapable generation of mist due to aging degradation or breakage caused by fatigue accumulation of the vibrating plate can be prevented.

Moreover, according to the aforementioned constitution, since presence of liquid-contact with the vibrating plate itself located at an end of the liquid supply path is detected instead of a liquid level of the liquid storage tank located in the middle of the liquid supply path or electrical conductivity of the liquid retaining material (sponge, for example), by means of an innovative design such that the liquid storage tank or the liquid retaining material is removed and a liquid amount required for one mist generation cycle (several tens of seconds, for example) is supplied to the vibrating plate each time, situations such as fungi growth, generation of odor, deposition of calcium carbonate or the like due to leaving of the used liquid for a long time in the liquid storage tank or the liquid retaining material can be also prevented. In addition, according to each-time supply of a slight amount of a mist material liquid as above, by keeping a damping load of the vibrating plate caused by contact with water to a required minimum, reduction of power consumption required for mist generation can be also realized.

In the aforementioned mist generating device, the vibrating plate is not limited to a specific structure (having a doughnut shape with a metal thin film on one surface, for example) which will be described but vibrating plates with various conventional structures such as a vibrator itself constituted by sandwiching a piezoelectric material between a pair of driving electrodes (see Japanese Utility Model Registration Laid-Open No. 05-070592) or a metal tongue cantilever-supported by the aforementioned vibrator (see Japanese Patent Laid-Open No. 04-150968 and Japanese Patent No. 3744931) can be employed as the vibrating plate.

Moreover, as the liquid supply mechanism, not limited to an inclined gutter which will be described later, various conventional structures such as a mechanism for generating a mist by dripping a liquid stored in a liquid storage tank onto the vibrating plate in a horizontal posture through a tube with a flow regulating valve (Japanese Patent Laid-Open No. 04-150968 and Japanese Utility Model Registration Laid-Open No. 05-070592) or a mechanism for generating a mist from an upper surface side by supplying a liquid to a lower surface of the vibrating plate through a liquid retaining material such as a sponge placed on the lower surface of the vibrating plate with fine holes and having a substantially horizontal posture (Japanese Patent No. 3744931) and the like can be employed as the liquid supply mechanism.

As an embodiment, the protective operation may be an operation for prohibiting (inhibiting) vibration of the vibrating plate itself. That is, as the aforementioned protective operation, various means such as an alarm sound (a buzzer or a voice, for example) for prompting a user to fill the liquid, an alarm display (lighting or flashing of a lamp, character display, for example) can be considered, but by prohibiting (inhibiting) the vibration itself of the vibrating plate in parallel with them or independently, fatigue accumulation caused by idle vibration of the vibrating plate can be prevented more reliably.

As an embodiment, the liquid-contact detecting unit may include first and second detecting electrodes between which the liquid is filled only during a period when the liquid is in contact with the vibrating plate and determining means for determining presence of contact of the liquid with the vibrating plate on the basis of a change in impedance between the first detecting electrode and the second detecting electrode.

According to such constitution, since the impedance (or more specifically, an electric resistance value or capacitive reactance) between the first electrode and the second electrode is largely changed between a state in contact with the liquid and a state not in contact with the liquid, presence of contact with the liquid can be reliably detected based on the change.

As an embodiment, it may be so configured that, by giving the same potential to both ends of a series circuit of the impedance between the both electrodes and a pull resistance at non-detection, while giving a known potential difference to the both ends of the series circuit at detection, the impedance is detected through a voltage drop generated in the pull resistance at that time.

According to such constitution, by forming the first detecting electrode and the second detecting electrode by different types of metal, even when a battery is constituted upon their contact with water, presence of contact of water with the vibrating plate can be reliably detected without being affected by an electromotive force of the battery.

At this time, if the first detecting electrode and the second detecting electrode are both made of bare conductors and the impedance is an electric resistance value, the electric resistance value between the first electrode and the second electrode is largely changed between the state with liquid-contact and the state without liquid-contact and thus, by fixing either one of the two detecting electrodes to a grounding potential or a power source potential, while by fixing the other to the power source potential or the grounding potential through a pull resistance, presence of the liquid-contact with the vibrating plate can be easily detected only by determining a potential change at the pull point through an appropriate comparator in a hardware or software manner.

On the other hand, if at least either one of the first detecting electrode and the second detecting electrode is made of a conductor having a thin dielectric film on a surface and the impedance is the capacitive reactance, the capacitive reactance between the first electrode and the second electrode is largely changed between the state with liquid-contact and the state without liquid-contact and thus, either one of the two detecting electrodes is fixed to the grounding potential or the power source potential, while the other is fixed to the power source potential or the grounding potential through the pull resistance, the capacitive reactance between the electrodes is reset and then, charging time until the potential of the pull point reaches a reference potential is determined through a comparator and a timer in a hardware or software manner so that presence of contact of the liquid with the vibrating plate can be easily detected.

In addition, those detecting presence of liquid-contact by using this change in the capacitive reactance have a merit that presence of contact of water with the vibrating plate can be reliably detected even under an environment where the conductor metal constituting the electrode is exposed to a mist material liquid and causes electric corrosion, and defective conduction can be easily generated.

In an embodiment, the liquid supply mechanism may have an injected liquid guiding portion for guiding a slight amount of the liquid injected or dripped through an inlet to the vibrating plate and a slight-amount liquid holding portion for holding the slight amount of the liquid guided by the injected liquid guiding portion in a state in contact with the vibrating plate until it is completely consumed by a mist generating action.

According to such constitution, by employing an innovative design that the slight amount of liquid required for one mist generation cycle (several tens of seconds, for example) is supplied each time to the vibrating plate, a surplus liquid remains in the liquid supply mechanism, and occurrence of the problem such as fungi growth, generation of odor, deposition of calcium or the like can be prevented.

At this time, if the slight-amount liquid holding portion is to hold the liquid in the state in contact with the vibrating plate by using a surface tension of the liquid, holding of the slight amount of liquid by the vibrating plate can be realized efficiently.

According to an embodiment, the vibrating plate is a vibrating plate with fine holes in which either one of front and rear surfaces is a liquid contact surface, while the other surface is a mist emission surface and arranged in a posture with the mist emission surface directed upward, the injected liquid guiding portion is an inclined gutter arranged so that its upstream end is located at the liquid inlet, while a downstream end is located on a lower side of the vibrating plate with fine holes, and the slight-amount liquid holding portion may be a narrow gap formed between the lower surface of the vibrating plate with fine holes and an upper surface of a gutter floor of the inclined gutter.

According to such constitution, by using the surface tension and/or a suctioning force (negative pressure) accompanying atomization of the liquid, holding of the slight amount of liquid can be realized with a simpler structure.

At this time, if the vibrating plate with fine holes is a piezoelectric vibrating plate formed by sequentially laminating and integrating a metal thin plate having fine holes, an annular first driving electrode, an annular piezoelectric material layer, and an annular second driving electrode and by insulating/covering a periphery of the metal thin plate while leaving the front and the rear, the first detecting electrode is the metal thin plate, and the second detecting electrode is a projection-shaped electrode provided on a floor surface of the gutter, accurate adhesion of the slight amount of liquid required for one mist generation cycle can be performed appropriately by using the surface tension and/or the suctioning force (negative pressure) of the atomization of the liquid, and a process from its emergence to loss can be reliably detected through the two electrodes.

The present disclosure when seen from another aspect can be grasped as a mist generating device having a liquid supply completion notifying function. That is, this mist generating device has a vibrating plate which vibrates at a high frequency and a liquid supply mechanism for supplying a conductive liquid such as water to the vibrating plate and is a mist generating device for generating a mist by bringing the liquid supplied through the liquid supply mechanism into contact with the vibrating plate for atomization and further includes liquid-contact detecting unit for detecting presence of liquid-contact with the vibrating plate; and notification operation performing unit for performing a notification operation for notifying completion of the liquid supply when the liquid-contact detecting unit detects a change from non-contact to contact of the liquid with the vibrating plate.

According to such constitution, during liquid supply, a user can confirm completion of the liquid supply on the basis of the notification operation.

At this time, if the notification operation is an operation for notifying completion of the liquid supply operation through generation of a mist by vibrating the vibrating plate in a predetermined mode, completion of the liquid supply operation can be known more reliably on the basis of the generation of the mist.

According to an embodiment, the mist generating device having the aforementioned various embodiments can be widely employed in various toys performing effects of smoke or water smoke (a steam locomotive toy ejecting smoke from a funnel, an automobile toy blowing out smoke from an exhaust pipe, a water fountain toy blowing up water smoke and the like).

The present disclosure when seen from another aspect can be more specifically grasped as a steam locomotive toy ejecting smoke from a funnel. This steam locomotive toy incorporates a mist generating device having a vibrating plate which vibrates at a high frequency and a liquid supply mechanism for supplying a conductive liquid such as water to the vibrating plate and generating a mist by bringing the liquid supplied through the liquid supply mechanism into contact with the vibrating plate for atomization inside an outer shell copying an appearance of a steam locomotive and performs an effect of smoke by discharging the mist generated in the mist generating device to an outside through a funnel provided on the outer shell, and further includes liquid-contact detecting unit for detecting presence of contact of the liquid with the vibrating plate; and protective operation performing unit for performing a protective operation for preventing idle vibration of the vibrating plate when the liquid-contact detecting unit detects no contact of the liquid with the vibrating plate.

According to such constitution, by preventing defective generation or incapable generation of mist due to aging degradation or breakage of the vibrating plate through the original excellent working effect of the aforementioned mist generating device, the highly reliable steam locomotive toy capable of stably maintaining the smoke ejecting function from the funnel can be realized.

At this time, if the protective operation is an operation of prohibiting (inhibiting) vibration of the vibrating plate itself even if a spraying instruction is given, the steam locomotive toy with higher reliability can be realized through the original excellent working effect in the embodiment of the aforementioned mist generating device.

In an embodiment, the liquid-contact detecting unit may include first and second detecting electrodes between which the liquid is filled only during a period when the liquid is in contact with the vibrating plate and determining unit for determining presence of contact of the liquid with the vibrating plate on the basis of a change in electric characteristics between the first detecting electrode and the second detecting electrode.

According to such constitution, through the original excellent working effect in the corresponding embodiment of the aforementioned mist generating device, a highly reliable steam locomotive toy capable of stably maintaining the smoke ejecting function from the funnel can be realized.

As an embodiment, it may be so configured that, by giving the same potential to both ends of a series circuit of the impedance between the both electrodes and a pull resistance at non-detection, while giving a known potential difference to the both ends of the series circuit at detection, the impedance is detected through a voltage drop generated in the pull resistance at that time.

According to such constitution, by forming the first detecting electrode and the second detecting electrode by different types of metal, even when a battery is constituted upon their contact with water, presence of contact of water with the vibrating plate can be reliably detected without being affected by an electromotive force of the battery.

In an embodiment, the liquid supply mechanism may have an injected liquid guiding portion for guiding a slight amount of the liquid injected or dripped through an inlet provided in the outer shell to the vibrating plate and a slight-amount liquid holding portion for holding the slight amount of the liquid lead by the injected liquid guiding portion in a state in contact with the vibrating plate until it is completely consumed by a mist generating action.

According to such constitution, a highly reliable steam locomotive toy capable of stably maintaining the smoke ejecting function from the funnel for a long time can be realized through an excellent working effect (electric corrosion measure) in the corresponding embodiment of the aforementioned mist generating device.

At this time, if the slight-amount liquid holding portion is to hold the liquid in a state in contact with the vibrating plate by using the surface tension of the liquid, the slight-amount liquid holding portion can efficiently realize holding of the slight amount of liquid by the vibrating plate.

In an embodiment, the vibrating plate is a vibrating plate with fine holes in which either one of front and rear surfaces is a liquid contact surface, while the other surface is a mist emission surface and arranged in a posture with the mist emission surface directed upward, the injected liquid guiding portion is an inclined gutter arranged so that its upstream end is located at the liquid inlet, while a downstream end is located below the vibrating plate with fine holes, and the slight-amount liquid holding portion may be a narrow gap formed between the lower surface of the vibrating plate with fine holes and an upper surface of the gutter floor of the inclined gutter.

According to such constitution, a highly reliable steam locomotive toy capable of stably maintaining the smoke ejecting function from the funnel for a long time can be realized through an excellent working effect in the corresponding embodiment of the aforementioned mist generating device.

At this time, the vibrating plate may be a piezoelectric vibrating plate formed by sequentially laminating and integrating the metal thin plate having fine holes for atomization, the annular first driving electrode, the annular piezoelectric material layer, and the annular second driving electrode and by insulating/covering the periphery of the metal thin plate while leaving the front surface of the metal thin plate, the metal thin plate side is fixed in a posture directed upward, the liquid supply mechanism includes a liquid inlet opened in an upper part of the outer shell and the inclined gutter for guiding the liquid injected through the liquid inlet to a lower surface side of the piezoelectric vibrating plate without storing it in the middle, a narrow gap for promoting capture of entry of the liquid by the surface tension is provided between a lower surface of the piezoelectric vibrating plate and an upper surface of the inclined gutter located below the piezoelectric vibrating plate, the first detecting electrode is made of the metal thin plate, and the second detecting electrode includes a projection-shaped electrode protruding from the upper surface of the inclined gutter toward the lower surface of the piezoelectric vibrating plate.

According to such constitution, by injecting or dripping the slight amount of the mist material liquid (water which is a conductive liquid, for example) required for one mist generation cycle (several tens of seconds corresponding to one smoke blowing-out running cycle of a steam locomotive toy, for example) through the liquid inlet arranged on the upper part of the outer shell, the slight amount of liquid injected or dipped as above has its upstream portion guided to the inclined gutter located immediately below the liquid inlet and is carried to the vicinity of the downstream end.

Between the gutter floor upper surface in the vicinity of the downstream end and the lower surface of the piezoelectric vibrating plate covering it, a narrow gap for promoting entry of the liquid by surface tension is provided. Therefore, the slight amount of liquid having reached the vicinity of the downstream end is filled in the gap by the surface tension and adheres/is captured by upper and lower wall surfaces in that state.

At this time, since the adhering/captured slight amount of liquid is brought into contact with a lower surface of a center region of the metal thin plate having a large number of fine holes (micron size) stacked thereon through a center hole of an annular laminated body formed by laminating and integrating three layers, that is, the annular first driving electrode, the annular piezoelectric material layer, and the annular second driving electrode, by means of a liquid atomizing action by high-frequency vibration of the metal thin plate, a mist made of liquid particles permeating the metal thin plate from a lower side to an upper side through the fine holes rises from the upper surface of a center part of the metal thin plate.

The metal thin plate also functions as the first detecting electrode. As a result, the material liquid is brought into contact with the first detecting electrode. On the other hand, when the slight amount of liquid adheres/is captured in the gap, this slight amount of liquid is also brought into contact with the projection-shaped electrode (second detecting electrode) protruding from the upper surface of the gutter floor.

As described above, in an initial state of the mist generating action when a slight amount of the material liquid is filled between the lower surface of the piezoelectric vibrating plate and the upper surface of the gutter floor, the first detecting electrode (metal thin plate) and the second detecting electrode (projection-shaped electrode) are both in contact with the material liquid (water). At the same time, the first detecting electrode and the second detecting electrode are electrically conducted also through the material liquid (water).

When the mist generating action has advanced, the amount of the material liquid decreases, and the amount no longer fills the gap, the material liquid collection (water droplet) leaves the upper surface of the gutter floor and adheres/is held on the lower surface of the piezoelectric vibrating plate through a negative pressure. In this state, the first detecting electrode (metal thin plate) and the second detecting electrode (projection-shaped electrode) are still in contact with the material liquid (water). At the same time, the first detecting electrode and the second detecting electrode are electrically conducted also through the material liquid (water).

When the mist generating action has further advanced, and a size of the material liquid collection (water droplet) decreases, the material liquid collection (water droplet) disappears in the end, but immediately before that, contact of the first detecting electrode (metal thin plate) and the second detecting electrode (projection-shaped electrode) with the material liquid is shut off, and at the same time, electric conduction between the first detecting electrode and the second detecting electrode is also disconnected.

Therefore, by monitoring a change in the electric characteristics (electric resistance value or static capacitance value, for example) between the first detecting electrode and the second detecting electrode, presence of contact of the mist material liquid with the piezoelectric vibrating plate can be accurately determined.

As a simple circuit for monitoring the electric characteristics between the first detecting electrode and the second detecting electrode, there can be a circuit in which the first detecting electrode is fixed to the grounding potential (GND) or the power source potential (Vcc), while the second detecting electrode is pulled up or down to the power source potential or the grounding potential through a resistive element so that potential variation at a pull-up point or a pull-down point is determined by comparison processing in a hardware or software manner.

In the aforementioned piezoelectric vibrating plate, too, by configuring a circuit so that the first driving electrode is at the grounding potential or the power source potential, the potential of the metal thin plate (first detecting electrode) conducted with that can be also fixed to the grounding potential or the power source potential.

Thus, according to the mist generating device including the aforementioned piezoelectric vibrating plate, since the first detecting electrode itself has been already fixed to the grounding potential or the power source potential, only by pulling up or pulling down the projection-shaped electrode which is the second detecting electrode to the power source potential or the grounding potential through the resistance, a circuit for detecting liquid-contact can be easily realized.

The present disclosure when seen from another aspect can be also grasped as a steam locomotive toy having a liquid-supply completion notifying function. That is, this steam locomotive toy is a mist generating device having a vibrating plate which vibrates at a high frequency and a liquid supply mechanism for supplying a conductive liquid such as water to the vibrating plate and generating a mist by bringing the liquid supplied through the liquid supply mechanism into contact with the vibrating plate for atomization and further includes liquid-contact detecting unit for detecting presence of contact of the liquid with the vibrating plate; and notification operation performing unit for performing a notification operation for notifying completion of the liquid supply when the liquid-contact detecting unit detects a change from non-contact to contact of the liquid with the vibrating plate.

According to such constitution, during liquid supply, a user can confirm completion of the liquid supply on the basis of the notification operation.

At this time, if the notification operation is an operation for notifying completion of the liquid supply operation through generation of a mist by vibrating the vibrating plate in a predetermined mode, completion of the liquid supply operation can be known more reliably on the basis of the generation of the mist.

The present disclosure when seen from another aspect can be also grasped as a steam locomotive toy operated by giving a slight amount of the mist material liquid corresponding to one smoke blowing-out running cycle (several tens of seconds, for example) each time. In this state, the fatigue accumulation caused by idle vibration of the vibrating plate can be solved by another method such as stopping vibration of the vibrating plate when a vibration time integrated value has reached a specified maximum value or the like.

That is, this steam locomotive toy is a steam locomotive toy which incorporates the mist generating device having the vibrating plate which vibrates at a high frequency and the liquid supply mechanism for supplying a conductive liquid such as water to the vibrating plate and generating a mist by bringing the liquid supplied through the liquid supply mechanism into contact with the vibrating plate for atomization inside the outer shell copying the appearance of the steam locomotive and performs an effect of smoke by discharging the mist generated in the mist generating device to an outside through a funnel provided on the outer shell, and the liquid supply mechanism has an injected liquid guiding portion for guiding a slight amount of the liquid injected or dripped through an inlet provided in the outer shell to the vibrating plate and a slight-amount liquid holding portion for holding the slight amount of the liquid guided by the injected liquid guiding portion in a state in contact with the vibrating plate until it is completely consumed by a mist generating action.

According to such constitution, by performing the subsequent injection each time the slight amount of the mist material liquid required for one smoke ejection running cycle is injected and this is completely consumed, for example, problems such as fungi growth caused by the remaining unused liquid, generation of odor, deposition of calcium on the inside or the like can be prevented.

At this time, if the slight-amount liquid holding portion is to hold the liquid in a state in contact with the vibrating plate by using the surface tension of the liquid, the slight-amount liquid holding portion can efficiently realize holding of the slight amount of liquid by the vibrating plate.

Moreover, if the vibrating plate is a vibrating plate with fine holes in which either one of front and rear surfaces is a liquid contact surface, while the other surface is a mist emission surface and arranged in a posture with the mist emission surface directed upward, the injected liquid guiding portion is an inclined gutter arranged so that its upstream end is located at the liquid inlet, while a downstream end is located below the vibrating plate with fine holes, and if the slight-amount liquid holding portion is a narrow gap formed between a lower surface of the vibrating plate with fine holes and an upper surface of a gutter floor of the inclined gutter, the highly reliable steam locomotive toy capable of stably maintaining a smoke ejecting function from the funnel for a long time can be realized through the excellent working effect (electric corrosion measure) in the corresponding embodiment of the aforementioned mist generating device.

The present disclosure when seen from another aspect can be also grasped as a steam locomotive toy system having a specific constitution. That is, this steam locomotive toy system includes a track and any one of the aforementioned series of steam locomotive toys, and at the railway station, a liquid injection facility having a liquid injection nozzle for injecting a slight amount of the liquid into a liquid inlet of the steam locomotive toy stopped at the railway station by a predetermined liquid injecting operation is provided.

According to such constitution, by means of a system configuration such that only the slight amount of the mist material liquid corresponding to one smoke ejection running cycle determined in advance is held in a vehicle body and each time the liquid is completely consumed, it is supplied at the railway station, unlike a case where a liquid storage tank is provided in the middle of the liquid supply path so as to supply the liquid to the vibrating plate via a tube therefrom or a liquid retaining material such as sponge is brought into contact with the vibrating plate, a surplus mist material liquid is not held inside the vehicle body and thus, situations such as fungi growth, generation of odor, deposition of calcium carbide or the like caused by leaving of the unused liquid in the liquid storage tank or the liquid retaining material for a long time can be prevented.

In an embodiment, in the outer shell of the steam locomotive toy, an outlet for discharging the liquid overflowing from the vibrating plate to the outside may be provided, and a recess portion for storing the liquid flowing out of the outlet of the stopped steam locomotive toy may be provided at the railway station on the track.

According to such constitution, the surplus mist material liquid overflowing through the gap can be prevented from remaining inside the vehicle body, and the mist material liquid discharged from the vehicle body can be also prevented from spreading over the surface of the floor in a play spot. At this time, if the recess portion is formed so as to present an appearance copying a pond, it can give more favorable appearance.

In the mist generating device, the steam locomotive toy, and the steam locomotive toy system described above, by providing the liquid-contact detecting unit for detecting presence of contact of the liquid to be a mist material with the vibrating plate, vibration of the vibrating plate is controlled, but it should be easily understood by those skilled in the art that the application of the liquid-contact detecting unit is not limited to that but can be widely applied to operation control in this type of mist generating devices.

According to the present disclosure, in a case where the liquid to be the mist material is not in contact with the vibrating plate for various reasons caused by the structure of the liquid supply mechanism such that the liquid storage tank is emptied, the liquid supply path from the liquid storage tank to the vibrating plate is clogged, the sponge which is the liquid retaining material is dried or the like, the protective operation for preventing idle vibration of the vibrating plate is immediately performed and as a result, defective generation or incapable generation of mist due to aging degradation or breakage caused by metal fatigue accumulation of the vibrating plate can be prevented.

Moreover, since presence of liquid contact of the vibrating plate itself located at an end of the liquid supply path is detected instead of a liquid level of the liquid storage tank located in the middle of the liquid supply path or electrical conductivity of the liquid retaining material (sponge, for example), by means of the innovative design such that the liquid storage tank or the liquid retaining material is removed and a liquid amount required for one mist generation cycle (several tens of seconds, for example) is supplied to the vibrating plate each time, situations such as fungi growth, generation of odor, deposition of calcium carbonate or the like due to leaving of the used liquid for a long time in the liquid storage tank or the liquid retaining material can be also prevented.

### BRIEF DESCRIPTION OF THE DRAWINGS

FIG. 1 is a configuration view of a steam locomotive toy system;

FIG. 2 is an appearance perspective view of the steam locomotive toy;

FIG. 3 is a perspective view illustrating major constituent elements inside the steam locomotive toy;

FIG. 4 is a side view illustrating the major constituent elements inside the steam locomotive toy;

FIGS. 5A-5C are action explanatory views (Part 1) of an injection portion;

FIGS. 6A and 6B are action explanatory views (Part 2) of the injection portion;

FIG. 7 is a sectional view illustrating a structure of a vibrator;

FIG. 8 is a circuit diagram (Part 1) schematically illustrating entire electric hardware configuration;

FIG. 9 is an explanatory view (Part 1) of a detection circuit;

FIGS. 10A-10C are waveform charts illustrating a signal state of each portion accompanying fluctuation of a water droplet size;

FIGS. 11A-11D are time charts explaining a relationship between a determination result of a detected voltage (Vx) and a driving signal (S1);

FIG. 12 is an explanatory view (Part 1) of the detection circuit;

FIGS. 13A and 13B are views illustrating a change in a voltage generated in an input port PI2 when an output port PO4 is switched to Vcc;

FIG. 14 is a view illustrating a voltage generated in the input port P12 when a detecting electrode is insulated;

FIG. 15 is a flowchart schematically illustrating an example of a control program of the steam locomotive toy;

FIG. 16 is a flowchart schematically illustrating an example of detected voltage determining processing; and

FIG. 17 is a flowchart schematically illustrating another example of detected voltage determining processing.

### DETAILED DESCRIPTION

An embodiment of a mist generating device, a steam locomotive toy, and a steam locomotive toy system according to the present disclosure will be described below in detail by referring to the attached drawings.

### <Steam Locomotive Toy System>

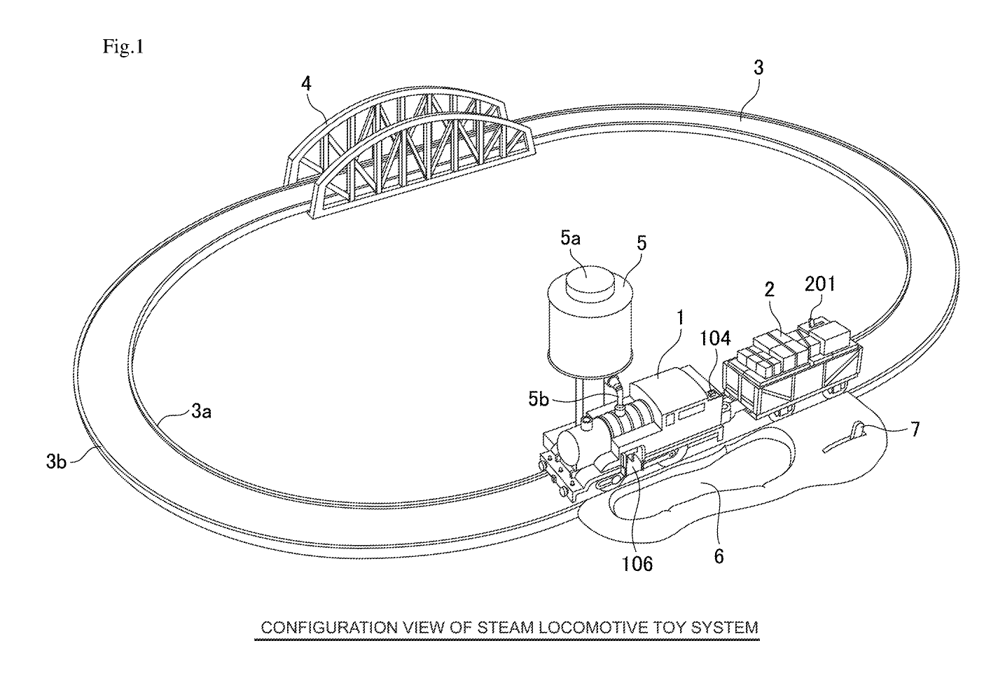

A configuration view illustrating an example of a steam locomotive toy system is illustrated in FIG. 1. As illustrated in the figure, this steam locomotive toy system mainly includes a steam locomotive toy 1 constituted as a head vehicle, a freight vehicle toy 2 connected to that and constituted as a second vehicle, a track 3 on which they run, and a railway station (details will be described later) provided on the track 3. Reference numerals 3a and 3b denote right and left guiding projections of the track 3 and reference numeral 4 denotes a bridge portion.

The steam locomotive toy 1 is constituted, in this example, as a non-power vehicle with no running power system such as a driving motor, a speed reduction gear train or the like incorporated. On the other hand, the freight vehicle toy 2 is constituted as a power vehicle incorporating a running power system such as a driving motor, a speed reduction gear train and the like, and by setting an operation lever 201 to either one of a front position and a rear position, one of a high-speed running and a low-speed running can be selectively performed. Thus, the steam locomotive toy 1 is capable of advancing at a high speed or a low speed by being pushed by the freight vehicle toy 2.

As described above, according to the constitution using the freight vehicle toy 2 as a power vehicle, a space in the steam locomotive toy 1 can be exclusively used for a device for mist generation or a device for generating sound or the like, and even when the freight vehicle toy 2 is removed and only the steam locomotive toy 1 is pushed manually for running, a smoke ejecting function, a sound generating function, and a light emitting function required as a steam locomotive can be effectively operated. However, the mist generating function according to the present disclosure can be also applied to a steam locomotive toy capable of self-powered running.

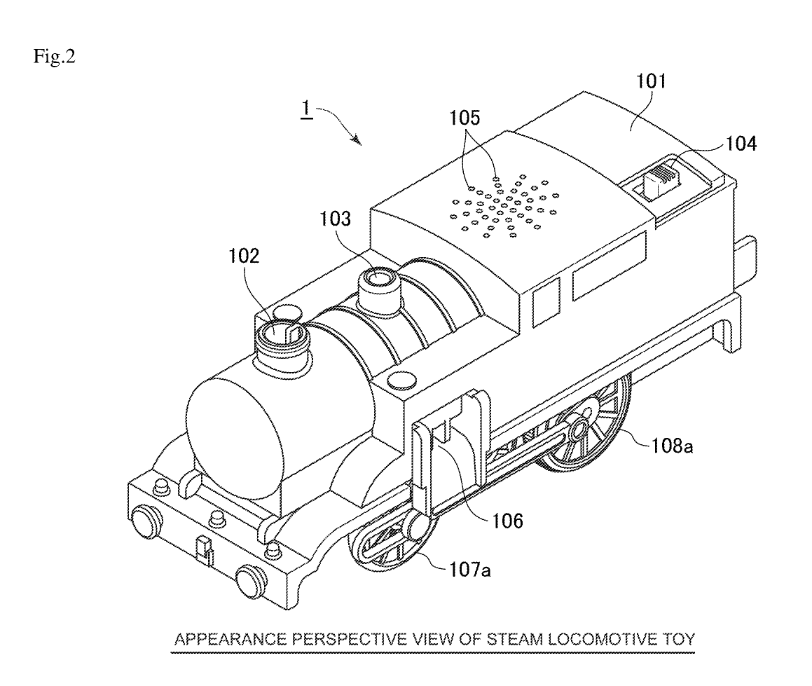

In the figure, a position where the steam locomotive toy 1 is drawn in the railway station. At this railway station, a water-supply facility 5 and a recess portion 6 copying a pond are provided so as to be located on sides opposite to each other with the track 3 between them. Though its internal mechanism is omitted, the water-supply facility 5 is constituted capable of injecting or dripping a slight amount of water stored inside into a water inlet (reference numeral 103 in FIG. 2) on the steam locomotive toy 1 side from a distal end of a water-supply nozzle 5b by pressing an operation button 5a.

The recess portion 6 copying a pond so as to be blended in a peripheral background is for receiving and storing surplus water discharged from a water outlet 106 provided on a side surface of the steam locomotive toy 1 when it is stopped at the railway station. Though details will be described later, a narrow gap or a cavity for catching a slight amount of water injected or dripped through the water inlet 103 by using surface tension and a negative pressure suctioning action is provided inside the steam locomotive toy 1, and the water that cannot be caught here is discharged as the surplus water through the water outlet 106. In the figure, reference numeral 7 denotes a lever for controlling stop-and-go of the freight vehicle toy 2 by elevating a center part on a track surface, not shown.

### <Appearance and Internal Structure of Steam Locomotive Toy>

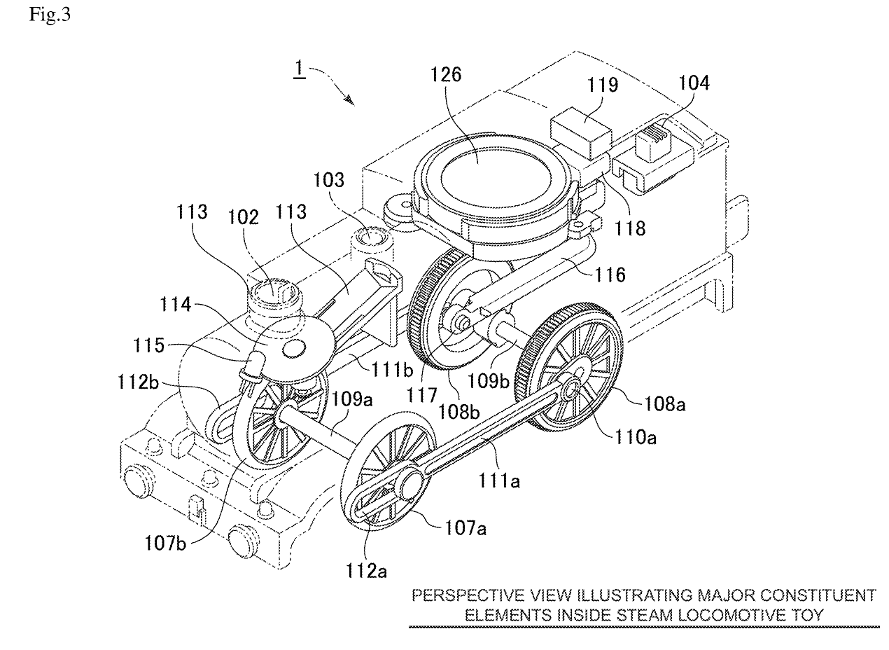

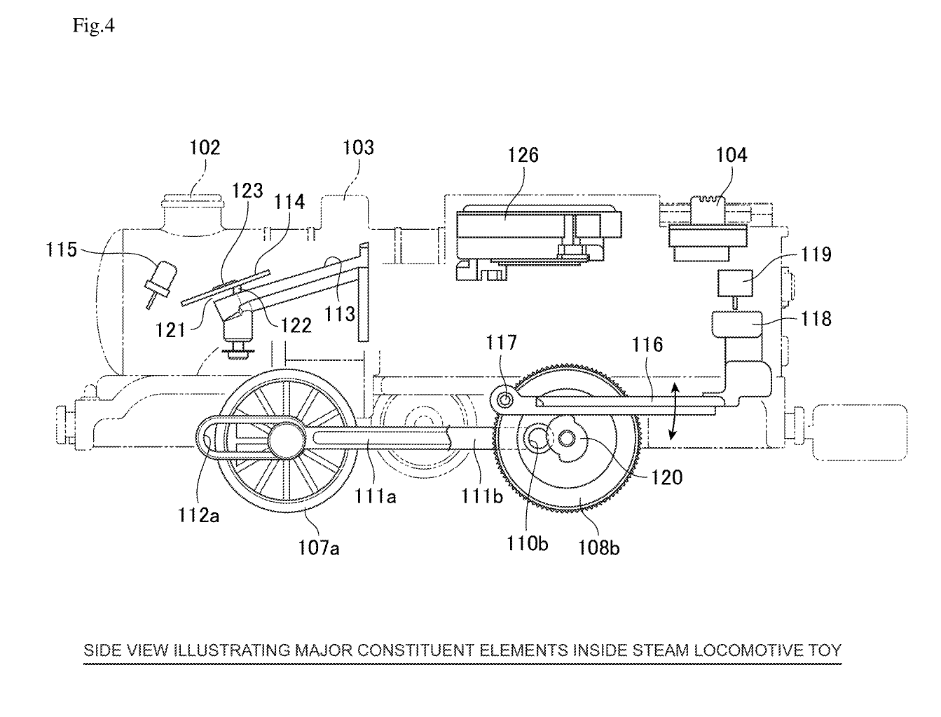

An appearance perspective view of the steam locomotive toy is illustrated in FIG. 2, and a perspective view and a side view illustrating major constituent elements inside thereof are illustrated in FIGS. 3 and 4, respectively. As illustrated in FIG. 2, the steam locomotive toy 1 has an outer shell 101 copying an appearance of a steam locomotive. On an upper surface of this outer shell 101, a funnel 102, the water inlet 103, a power switch 104, and sound emission holes 105 for emitting sound of a built-in speaker (reference numeral 126 in FIG. 3) to an outside are provided. Moreover, on the side surface of the outer shell 101, the water outlet 106 for discharging the surplus water described above is provided.

As illustrated in FIGS. 3 and 4, the outer shell 101 includes therein: 1) components required for running on the track (hereinafter, referred to as “running components”); 2) components for generating a vehicle-speed pulse in conjunction with rotation of a wheel (hereinafter, referred to as a “vehicle-speed pulse generating components”); 3) components for generating an effect sound (a Russel sound or human voices) or effect light (illumination of smoke) (hereinafter, referred to as “effect generating components”); 4) components for generating a mist which is an essential part of the present disclosure (hereinafter referred to as “mist generating components”); and 5) components for detecting liquid-contact. Those components will be sequentially described below.

1) Running Component

As the running components, left and right front wheels 107a and 107b, left and right rear wheels 108a and 108b, front and rear axles 109a and 109b, and left and right connecting rods 111a and 111b connecting the left and right front and rear wheels can be cited. The left and right connecting rods 111a and 111b are supported in rear end holes 110a and 110b rotatably to eccentric positions of the rear wheels 108a and 108b and also supported in front end long holes 112a and 112b slidably to center positions of the front wheels 107a and 107b. Thus, they are constituted such that a motion copying a piston motion specific to a steam locomotive in which the left and right rear wheels 108a and 108b are rotated/driven is presented by apparent expansion/contraction of the left and right connecting rods 111a and 111b.

2) Vehicle-Speed Pulse Generating Component

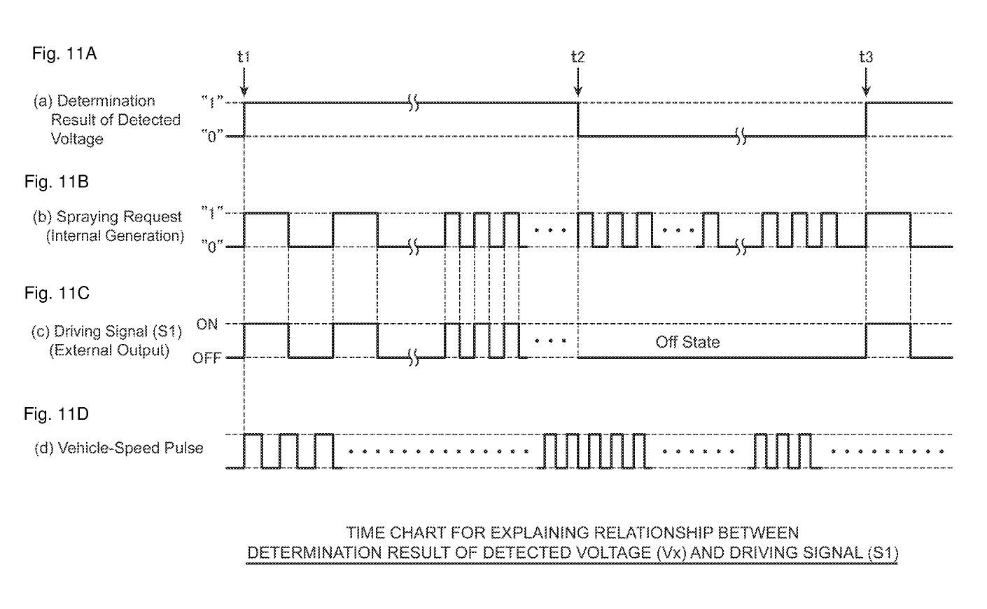

As the vehicle-speed pulse generating components, a lever 116 having a base end portion 117 journaled to a machine casing rotatably and capable of vertical swing using it as a fulcrum, a cam (see reference numeral 120 in FIG. 4) on which a cam surface in contact with a lower surface of the lever 116 is formed on its peripheral surface, an operator 118 mounted on a distal end portion of the lever 116 and elevated in conjunction with the swing of the lever 116, and a switch 119 outputting a vehicle-speed pulse (FIG. 11D) which is a series of pulse trains having a pulse interval synchronized with rotation of the wheels by intermittent on/off in conjunction with the elevation of the operator 118 can be cited. In the illustrated example, two pulses are generated from the switch 119 at each rotation of the left and right rear wheels 108a and 108b.

3) Effect Generating Component

As will be described above, the mist generated in a mist generating portion is emitted as a white smoke from the funnel 102 to the outside at emission timing determined on the basis of a vehicle-speed pulse. At this time, the mist passing through the funnel 102 is illuminated in an appropriate color (red, for example), and an effect as if light of a combustion furnace leaks out is performed. A light emission diode 115 is used as illuminating unit. Moreover, an effect sound corresponding to a Russel sound of a steam locomotive is generated and a talk sound corresponding to a human voice is also generated at sound emitting timing generated on the basis of the vehicle-speed pulse. The generation of these sounds is made through a speaker 126 and the Russel sound and the talk sound generated as above are emitted to the outside through the sound emission holes 105.

4) Mist Generating Component

As the mist generating components, a piezoelectric vibrating plate 114 functioning as a vibrator and an inclined gutter 113 for guiding a slight amount of water injected or dripped through the water inlet 103 to the piezoelectric vibrating plate 114 can be cited.

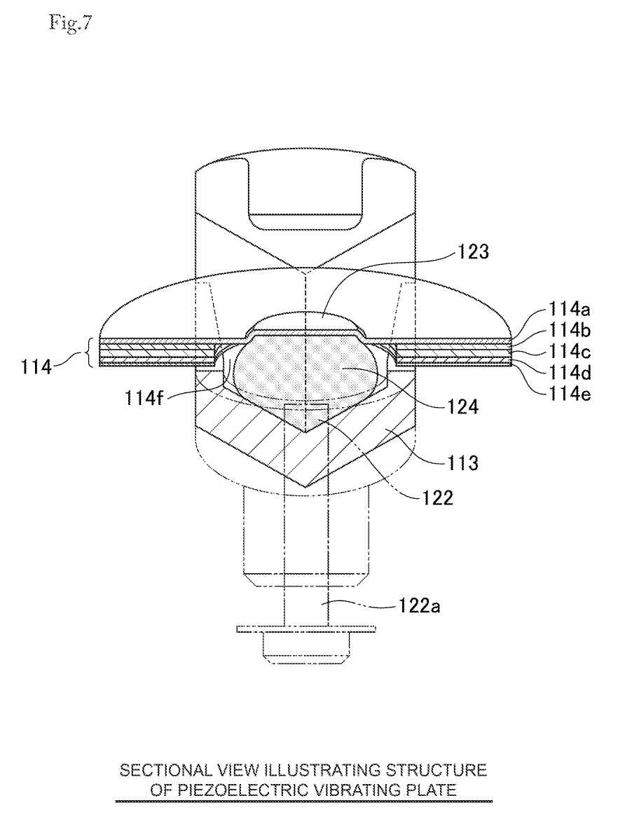

A sectional view illustrating a structure of the piezoelectric vibrating plate 114 is illustrated in FIG. 7. As illustrated in the figure, the piezoelectric vibrating plate 114 is constituted by laminating and integrating four elements, that is, a disc-shaped metal thin plate 114a made by using metal such as stainless or the like, an annular (doughnut-shaped) first driving electrode 114b made by using metal such as Ag or the like, an annular (doughnut-shaped) piezoelectric material layer 114c made by using a piezoelectric material such as ceramic or the like, and an annular (doughnut-shaped) second driving electrode 114d made by using metal such as Ag or the like and by covering a periphery (an inner circumference of a center hole 114f, an outer circumference of the four-element laminated body, and a lower surface of the second driving electrode) excluding a surface (an upper surface and a lower surface exposed to the center hole 114f) of the metal thin plate 114a with an insulating film 114e. This insulating film also contributes to corrosion resistance of the electrode 114d located on the lower surface of the piezoelectric vibrating plate 114. A small circular region 123 at the center part of the disc-shaped metal thin plate 114a is formed by slightly expanding to an upper surface side, and a large number of micron-sized fine holes are provided in this small circular region 123. Lead wires, not shown, are lead from the first and second driving electrodes 114b and 114d.

Thus, as will be described later, when the slight amount of water or a water droplet 124 which is a mist material is captured between the upper surface of the gutter floor in the inclined gutter 113 and the lower surface of the piezoelectric vibrating plate 114, this water droplet 124 passes through the center hole 114f of the piezoelectric vibrating plate 114, is brought into contact with the lower surface of the small circular region 123 in the metal thin plate 114a and is electrically conducted with that.

In this state, when a high-frequency voltage (110 kHz, for example) is applied between the first and second driving electrodes 114b and 114d, expansion/contraction of the piezoelectric material layer 114c is repeated at a high speed, and the vibrating plate 114 performs high-frequency vibration (resonance) at a high Q with a small loss. Then, the water 124 in contact with the lower surface of the small circular region 123 in the metal thin plate 114a is atomized by being permeated through the large number of fine holes provided in the small circular region 123 to the upper surface side, whereby a mist 125 is generated.

When the mist generating action is generated as above, a negative pressure is generated on the lower surface side of the small circular region 123, and the captured slight amount of water or water droplet 124 is made to adhere to the lower surface of the small circular region 123 more strongly and as a result, the captured slight amount of water or water droplet 124 firmly adheres to the lower surface of the small circular region 123 and continues to be in contact with that in combination with the surface tension until it is completely consumed by the mist generating action.

On the other hand, according to the constitution in which the water supplied to the piezoelectric vibrating plate 114 is brought into contact not with the whole surface of the piezoelectric vibrating plate 114 but only with the lower surface of the small circular region 123, electric power required for the atomizing action for the mist generation can be drastically reduced. That is, since the piezoelectric vibrating plate 114 is excited in a mechanical resonance state and large amplitude is obtained, but since it resonates at the high Q with a small loss, the amplitude can be easily affected by even slight damping caused by contact with the water. Thus, according to the constitution in which only the lower surface of the small circular region 123 is brought into contact with the water, spraying with low power consumption is realized by keeping a water-waving area to a required minimum.

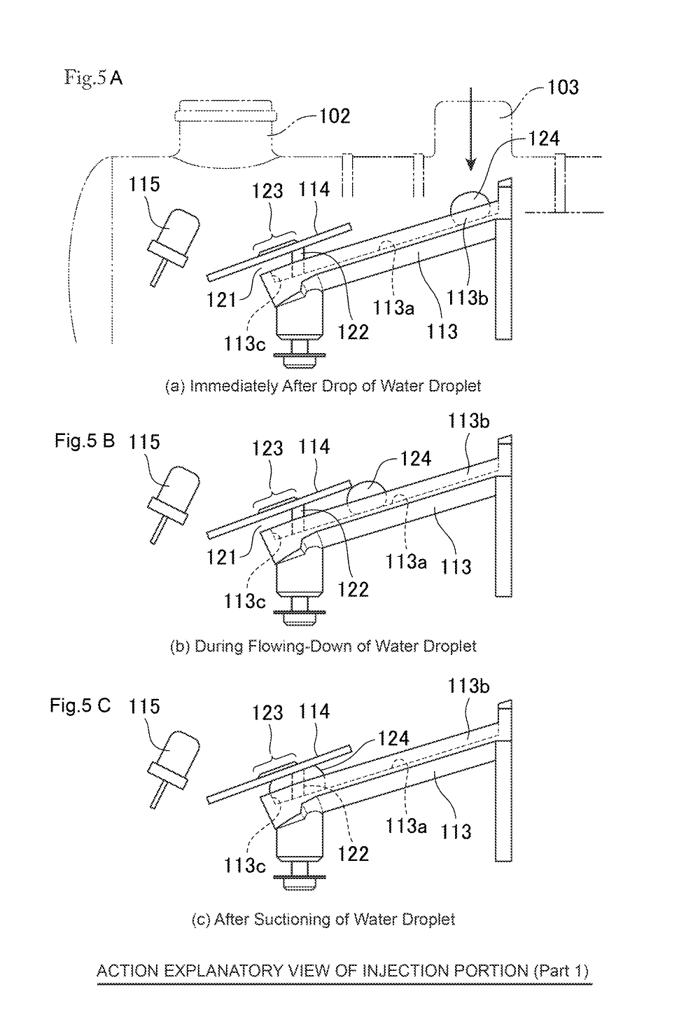

Returning to FIGS. 3 and 4, the inclined gutter 113 is a gutter having the gutter floor surface (reference numeral 113a in FIGS. 5A-5C) having a V-shaped section, and its upstream portion (reference numeral 113b in FIGS. 5A-5C) is located immediately below the water inlet 103, while the downstream portion is supported in an inclined posture so as to be located on the lower surface side of the piezoelectric vibrating plate 114. The surface of the gutter floor surface (reference numeral 113a in FIGS. 5A-5C) having a V-shaped section may be constituted with water-repellence so that the slight amount of water or water droplet injected or dripped through the water inlet 103 flows down smoothly toward the downstream. On a downstream end of the inclined gutter 113, a downstream end wall (reference numeral 113c in FIGS. 5A-5C) having a function of retaining the flowing-down water at the downstream end to some degree is provided.

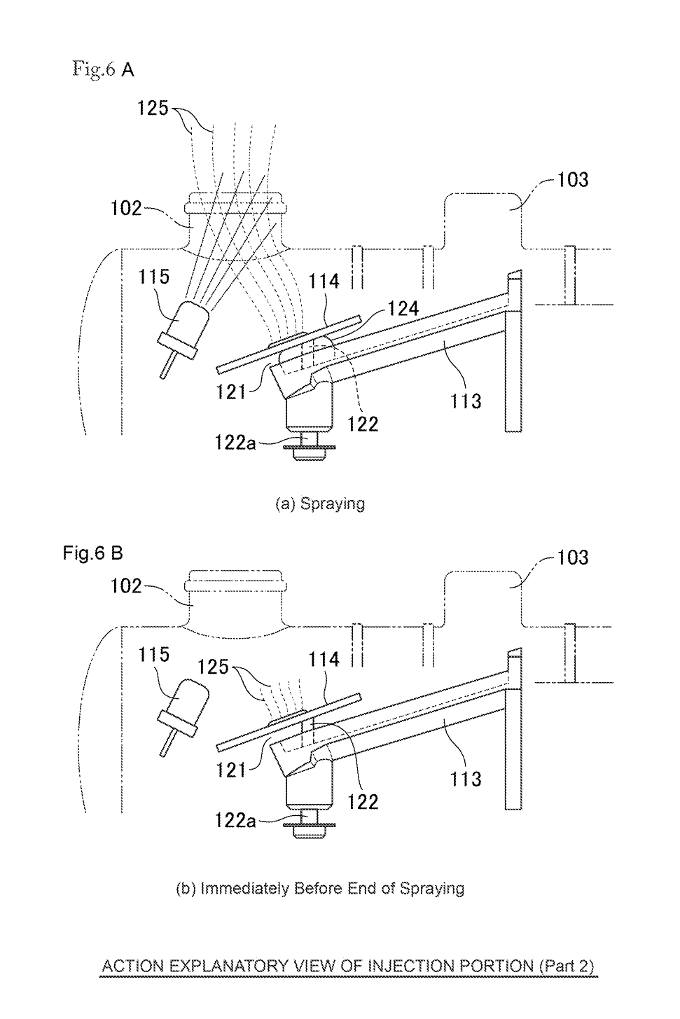

The piezoelectric vibrating plate 114 is, as illustrated in FIGS. 5 and 6, supported in a state with the metal thin plate 114a side directed upward and in this example, an inclined posture substantially in parallel with the gutter floor surface 113a of the inclined gutter 113 in accordance with an inclination angle of the inclined gutter 113. The parallelism between the inclined gutter 113 and the piezoelectric vibrating plate 114 is not indispensable in the present disclosure. An important point here is that a narrow gap 121 for promoting entry of the slight amount of water or water droplet 124 between the floor surface 113a of the inclined gutter 113 and the lower surface of the piezoelectric vibrating plate 114 is provided between them. When such narrow gap is present, the slight amount of water or water droplet 124 having reached the downstream of the inclined gutter 113 enters the gap 121 as if it is suctioned by its surface tension and adheres to upper and lower wall surfaces (the lower surface of the piezoelectric vibrating plate 114 and the upper surface of the gutter floor 113a) and the downstream end wall 113c and is captured on the spot.

5) Liquid-Contact Detecting Component

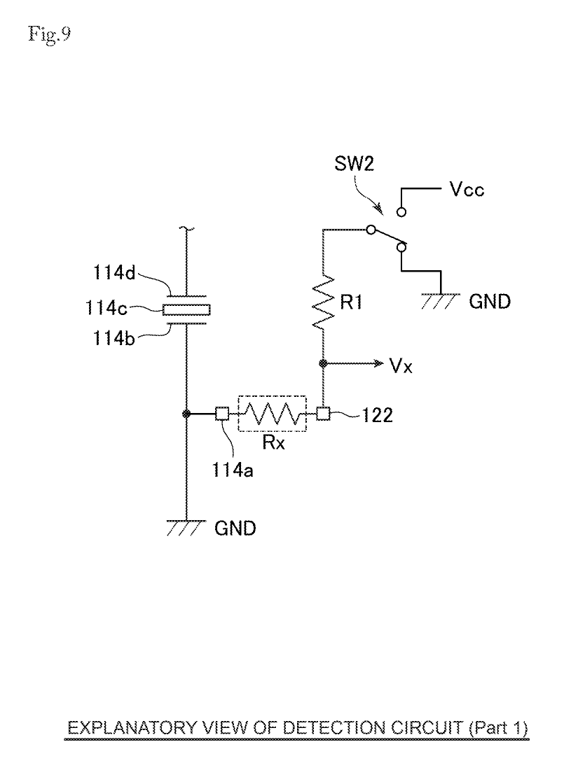

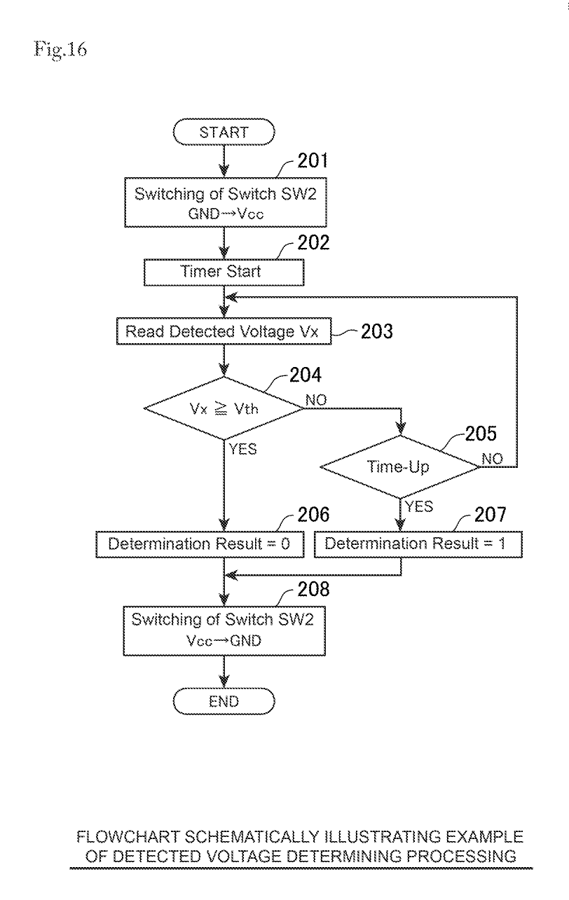

In order to detect whether or not the vibrating plate is in contact with the water or water droplet 124 which is a mist material, the first detecting electrode and the second detecting electrode between which is filled with water only when the water or water droplet 124 is in contact with the vibrating plate are needed. In this example, the metal thin plate (a thin plate made of stainless having a nickel-plated layer on the surface in this example) 114a itself constituting the piezoelectric vibrating plate 114 functions as the first detecting electrode. The metal thin plate 114a is electrically conducted with the first driving electrode 114b, and in this example, it has potential substantially fixed to the grounding potential (GND) (see FIG. 9). On the other hand, in this example, a projection-shaped electrode 122 protruding from the gutter floor surface 113a on the downstream portion of the inclined gutter 113 functions as the second detecting electrode. A slight gap may be present between a distal end of this projection-shaped electrode 122 and the piezoelectric vibrating plate 114. In this example, as the projection-shaped electrode 122 functioning as the second detecting electrode, a distal end portion of a screw 122a made of stainless and screwed from a lower side to an upper side is used (see FIG. 16). The distal end portion is separated from the lower surface of the piezoelectric vibrating plate 114 through a slight gap in this example.

### <Action of Mist Generating Portion>

Subsequently, an action of the mist generating portion constituted by the inclined gutter 113 and the piezoelectric vibrating plate 114 will be described by referring to FIGS. 5 and 6. An action explanatory view (Part 1) and the same (Part 2) of the mist generating portion are illustrated in FIGS. 5 and 6.

At the railway station, the slight amount of water or water droplet 124 injected or dripped to the water inlet 103 from a water-injection nozzle 5b first drops to the floor surface 113a of the upstream portion 113b in the inclined gutter 113 (see FIG. 5A). Subsequently, the slight amount of water or water droplet 124 flows down to the gutter floor surface 113a having a V-shaped section while being guided and reaches the vicinity of an edge part of the piezoelectric vibrating plate 114 (see FIG. 5B). The narrow gap 121 promoting entry of water by the surface tension is present between the piezoelectric vibrating plate 114 and the gutter floor surface 113a. Thus, the slight amount of water or water droplet 124 having reached an inlet of this gap 121 enters into the gap 121 as if it is suctioned by the surface tension and is captured on the spot by adhering to the upper and lower wall surfaces and the downstream end wall 113c (see FIG. 5C). At this time, as illustrated in FIG. 7, the slight amount of water or water droplet 124 is substantially contained in the center hole 114f of the piezoelectric vibrating plate 114 and enters a state in contact with the lower surface of the small circular region 123 located at the center of the metal thin plate 114a. In this state, when the piezoelectric vibrating plate 114 is driven, by means of the water atomizing action by the high-frequency vibration of the metal thin plate 114a having fine holes, the mist 125 is generated from the upper surface of the small circular region 123 of the piezoelectric vibrating plate 114. By means of emission of the mist 125 generated as above to the outside through the funnel 102, an effect of white smoke is performed, and at the same time, the light emission diode 115 is lighted or flashed, whereby the inside of the funnel 102 is illuminated in red, and an effect of leakage of light from a combustion chamber is performed (see FIG. 6A). Subsequently, as generation of the mist advances, an amount or a size of the slight amount of water or water droplet 124 decreases, and disappearance thereof finishes the mist generation (see FIG. 6B).

In the aforementioned series of processes, the slight amount of water or water droplet 124 filled in the center hole portion 114f of the gap 121 has its amount or size gradually decreased as the mist generation advances, and at a certain point of time and after, in combination with a negative pressure suctioning force accompanying the water atomizing action, it leaves the floor surface 113a and adheres to the lower surface of the vibrating plate 114, and while its amount or size is further decreasing in that state, it disappears in the end. On the other hand, a space between the first detecting electrode (metal thin plate 114a) and the second detecting electrode (projection-shaped electrode 122) starts electrical conduction at a point of time when the gap 121 is filled with the water droplet 124 and becomes non-conductive at a point of time immediate before the water droplet 124 disappears. Thus, by observing the electrical characteristics between the first detecting electrode and the second detecting electrode, presence of contact of the water droplet 124 with the vibrating plate 114 (to be more accurate, the lower surface of the small circular region 123 in the metal thin plate 114a) can be detected easily.

### <Electric Hardware Configuration>

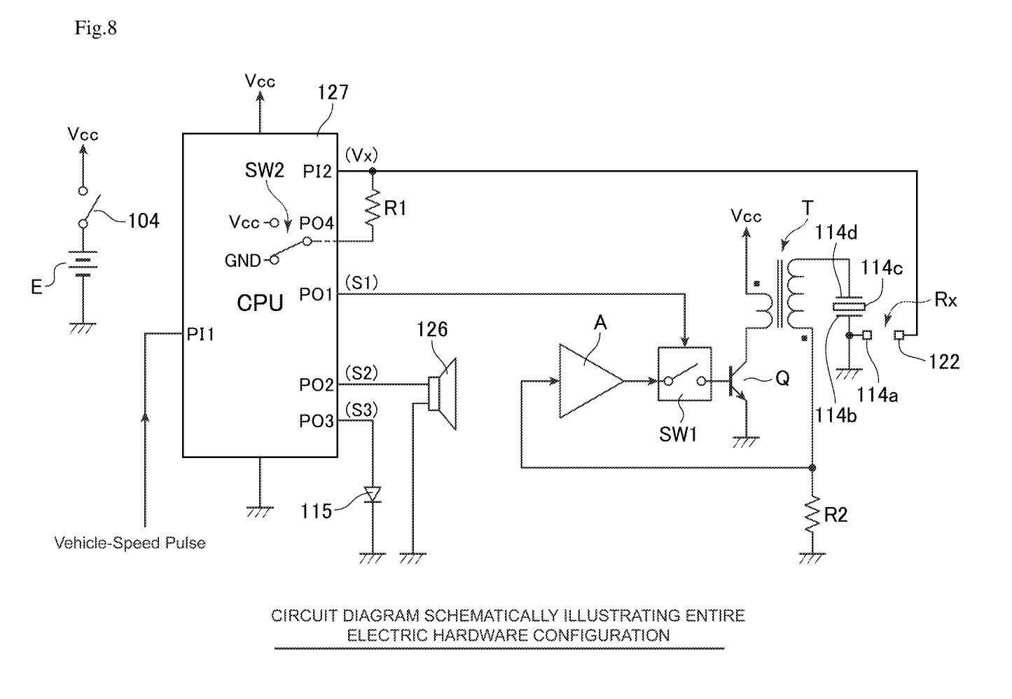

Subsequently, electric hardware configuration of the steam locomotive toy will be described. A circuit diagram schematically illustrating entire electrical hardware configuration is illustrated in FIG. 8. As illustrated in the figure, the entire electric circuit of the steam locomotive toy mainly includes a driving circuit (details will be described later) for resonating the piezoelectric vibrating plate 114 which is a vibrator at its natural frequency, a detection circuit (details will be described later) for detecting contact of water with the piezoelectric vibrating plat 114 which is a vibrator, and a CPU 127 for integrally controlling the entire steam locomotive toy. Reference character E denotes a power source and is constituted by connecting two AAA size cells in series, for example. Reference numeral 104 denotes a power switch for supplying power to the circuit.

1) Driving circuit

First, the driving circuit for resonating the piezoelectric vibrating plate 114 which is a vibrator at its natural frequency will be described. This driving circuit mainly includes an amplifier A, a boosting transformer T, and a driving transistor Q and is configured so as to function as a self-oscillation circuit in which a current circulating to the piezoelectric vibrating plate 114 which is a piezoelectric vibrator through the boosting transformer T is converted to a voltage through a slight resistance R2 and returned to the amplifier A. This self-oscillation circuit performs an oscillating operation at a resonance frequency (110 kHz, for example) of the piezoelectric vibrating plate 114 which is a piezoelectric vibrator. The piezoelectric vibrating plate 114 which is a piezoelectric vibrator is driven by a flyback voltage of the boosting transformer T and is vibrated at a high frequency, and a mist is generated by the water atomizing action in contact with that. This mist generation is intermittently continued as appropriate by on/off of a switch SW1 in response to a driving control signal S1 which is a pulse train sent from the CPU 127, which causes on/off of the transistor Q upon receipt of that.

2) Detection Circuit

Subsequently, the detection circuit for detecting contact of the water with the piezoelectric vibrating plate 114 will be described. This detection circuit is one (first detecting electrode) of a pair of detecting electrodes and which is the metal thin plate 114a fixed to the GND potential and the other of the pair of detecting electrodes and which is the projection-shaped electrode 122 connected to an output port PO4 of the CPU 201 through the pull resistance R1, an internal switch SW2 subjected to switching control through a program and leading either one of the Vcc potential and the GND potential to the output port PO4, and an input port PI2 for taking in the detected voltage Vx appearing at a connection point between the pull resistance R1 and inter-electrode resistance Rx as illustrated in FIGS. 8 and 9.

When the water detecting operation is not performed, the internal switch SW2 is connected to the GND side, and the GND potential appears at the output port PO4. Thus, the projection-shaped electrode 122 is forcedly pulled down to the GND potential, and the pair of electrodes 114a and 122 both are at the GND potential, and a potential difference is not generated between the both electrodes. At this time, the potential (detected voltage Vx) appearing at the input port PI2 is maintained at the GND potential whether the water is present between the both electrodes or not.

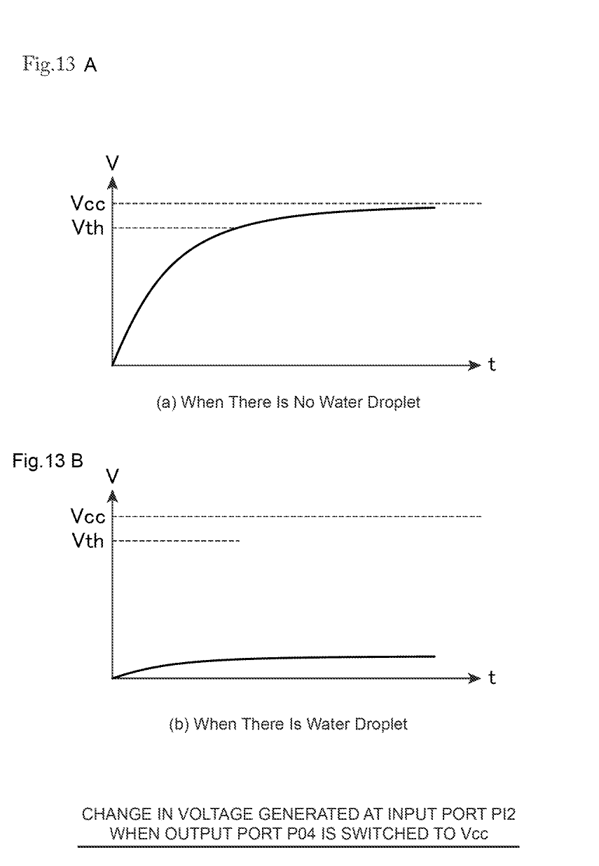

On the other hand, when the water detecting operation is to be performed, the internal switch SW2 is switched from the GND side to the Vcc side, and since the Vcc potential appears at the output port PO4, the projection-shaped electrode 122 is forcedly pulled up to the Vcc potential. Then, if there is no water between the both electrodes (when the water is not in contact with the vibrating plate 114), as illustrated in FIG. 13A, the potential (detected voltage Vx) appearing at the input port PI2 rapidly rises while drawing a predetermined time constant curve and exceeds a threshold value voltage Vth at a certain point of time. On the other hand, if there is water between the both electrodes (the vibrating plate 114 is in contact with the water), the potential (detected voltage Vx) appearing at the input port PI2 gently rises while drawing the predetermined time constant curve but does not exceed the threshold value voltage Vth. Thus, after the internal switch SW2 is switched from the GND side to the Vcc side, by comparing the value of the detected voltage Vx with the threshold value voltage Vth with some waiting time Tw, it can be determined whether the water is in contact with the vibrating plate 114 or not.

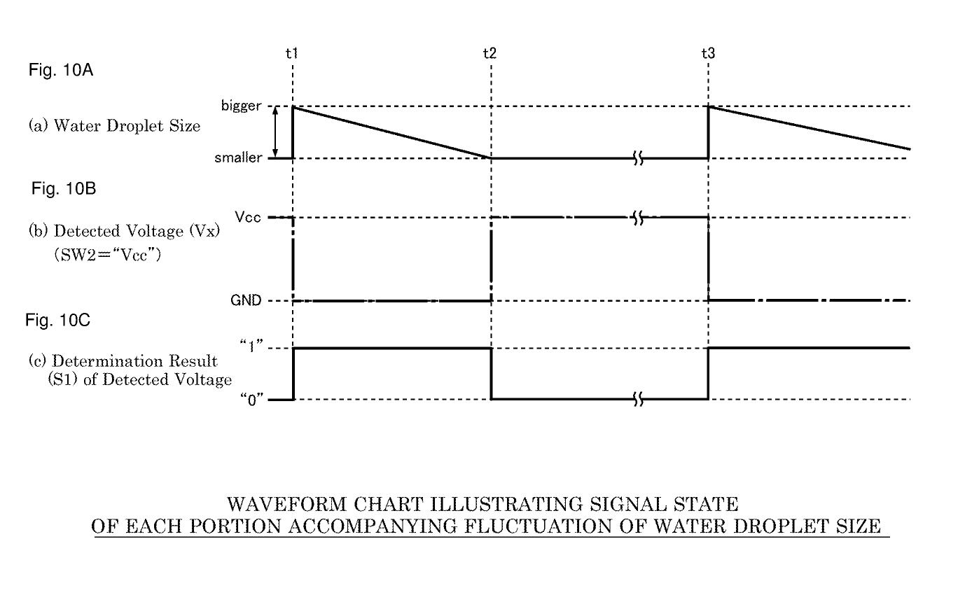

In the water detecting operation, a waveform chart illustrating a signal state of each portion accompanying fluctuation of the water droplet size is illustrated in FIGS. 10A-10C. Assuming that an appropriate amount of water is filled in the gap 121 between the piezoelectric vibrating plate 114 and the gutter floor 113 by injecting or dripping a slight amount of the water through the water inlet 103 at time t1 (see FIG. 10A, the value of the detected voltage Vx falls from the Vcc potential to the GND potential (see FIG. 10B), and at a time when the detected voltage Vx exceeds the threshold value Vth set in advance, a logical value of the detected voltage determination result changes from “0” to “1” (see FIG. 10C). After that, by means of continuation of the mist generating action, the water droplet size gradually decreases, and when the water droplet substantially disappears at time t2 (see FIG. 10A), the value of the detected voltage Vx rises from the GND potential to the Vcc potential (see FIG. 10B), and the logical value of the detected voltage determination result changes from “1” to “0” (see FIG. 10C). After that, until time t3 when the water is newly injected, the logical value of the detected voltage determination result is maintained in the “0” state (see FIG. 10C). As will be described later, vibration of the piezoelectric vibrating plate 114 is forcedly inhibited (prohibited) by the logical value “0” of this detected voltage determination result, and as a result, defective mist generation or incapable generation of the mist due to breakage by accumulation of metal fatigue caused by idle vibration of the piezoelectric vibrating plate 114 or the like is prevented.

The reason why the value of the detected voltage Vx rises by drawing the predetermined time constant curve as illustrated in FIGS. 13A and 13B immediately after the internal switch SW2 is switched from the GND side to the Vcc side is estimated to be caused by wiring capacitance from the input port PI2 of the CPU 127 to the electrode 122 and presence of capacitive reactance due to water interposed between the both electrodes. The larger the value of the pull resistance R1 is, the more favorable the detection sensitivity of the water becomes, but considering mis-detection due to a leak current, approximately 10 kΩ to 100 kΩ is preferable. The value of the aforementioned waiting time Tw is also different depending on the value of the pull resistance R1 and a wiring state but it can be set to approximately 100 μsec, for example.

Only when the water is to be detected, the internal switch SW2 is switched from the GND side to the Vcc side and the projection-shaped electrode 122 is pulled up to the Vcc potential because if the projection-shaped electrode 122 is kept in a state pulled-up to the Vcc potential at all times, in a case where the two electrodes 114a and 122 are made of metal of different types from each other, a potential is generated between the both electrodes due to ionization tendency, and the water detection is affected by that. According to an experiment by the inventors, in a case where one of the pair of electrodes (first detecting electrode) is the metal thin plate 114a which is stainless with the nickel-plated surface and the other (second detecting electrode) is the projection-shaped electrode 122 which is a screw distal end made of solid stainless, if the water is interposed between the both electrodes, a battery cell is constituted by the metal thin plate 114d as a negative electrode and the projection-shaped electrode 122 as a positive electrode and in addition, charging through the pull resistance R1 is made, and the potential of the projection-shaped electrode 122 gradually rises and exceeds the threshold value voltage Vth in the end, whereby nonconformity of mis-determination as water shortage can occur though water remains between the electrode.

3) CPU

Subsequently, the CPU 127 for integrally controlling the entire steam locomotive toy will be described. The CPU 127 includes a microprocessor, an ASIC having various dedicated functions, and a memory (ROM, RAM). In a CPU 201, in addition to terminals (Vcc, GND) for feeding power, at least an input port PI1 for taking in the vehicle-speed pulse, the input port PI2 for taking in the detected voltage Vx, an output port PO1 for outputting the driving signal S1 (details will be described later), an output port PO2 for outputting an audio signal S2 for driving the speaker 126, an output port PO3 for outputting a diode driving signal S3 for driving the light emission diode 115, and the output port PO4 for selectively outputting the GND potential and the Vcc potential in accordance with the switching of the internal switch SW2.

Here, the detected voltage Vx is, as described above, a voltage at the input port PI2 at a point of time when appropriate waiting time Tw (differed depending on the value of the resistance R1 or the wiring state to the electrode 122 but approximately 100 μsec, for example) has elapsed since the potential of the output port PO4 is switched from the GND potential to the Vcc potential and a voltage fluctuated between the GND potential and the Vcc potential in accordance with the value of electric resistance Rx (see FIG. 9) between the first detecting electrode (metal thin plate 114a in FIG. 7) and the second detecting electrode (projection-shaped electrode 122 in FIG. 7) (see FIG. 10B). In FIG. 10B, it should be noted that the value is indicated by a one-dot chain line, considering that the value is what appears each time the water detecting operation is performed and is not present at all time.

The driving signal S1 is a binary signal for controlling a state of the aforementioned driving circuit and is configured such that an oscillating state is instructed to the driving circuit when the output of the driving signal S1 is in an ON state, while an oscillation stopped state is instructed in the case of an OFF state, respectively (see FIG. 11C).

### <Electric Software Configuration>

1) Entire Processing

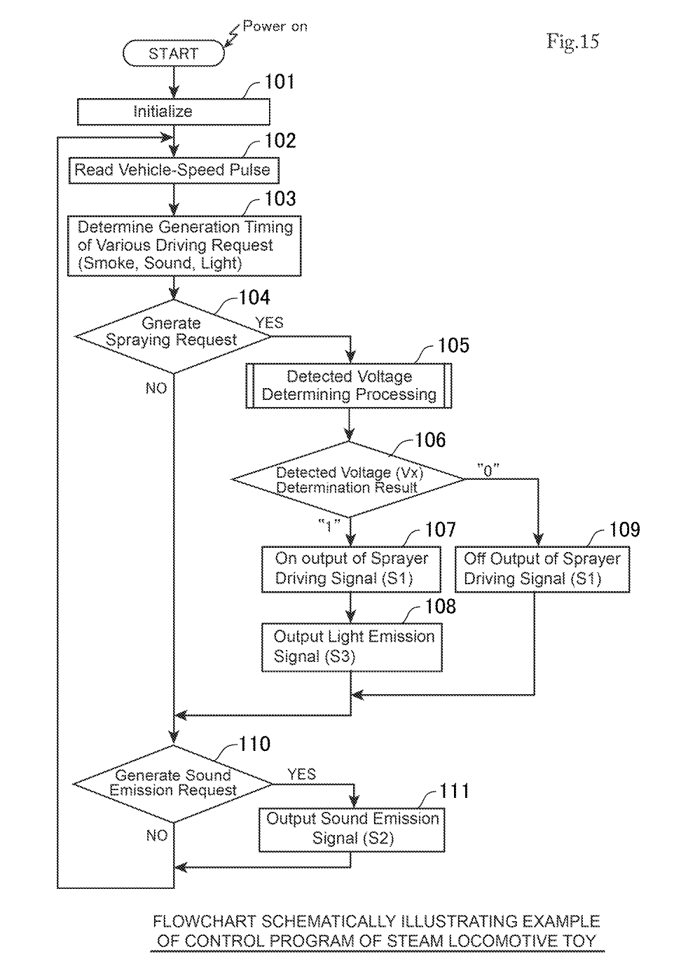

A flowchart schematically illustrating an example of a control program of the steam locomotive toy is illustrated in FIG. 15. In the figure, when processing is started by power on of the power switch 104, first, initial setting of various flags and registers is carried out by initializing processing (Step 101) and then, the vehicle-speed pulse is read from the input port PI1, and a generation mode of the vehicle-speed pulse (pulse generation timing, a pulse generation cycle, continuity of certain number of pulses and the like) is analyzed (Step 102). Then, on the basis of the aforementioned analysis result, generation timing of various generation requests (smoke, sound, light) is determined (Step 103). After that, while the reading processing of the vehicle-speed pulse (Step 102) and the generation timing determining processing (Step 103) are executed, internal generation of a spray generation request (see FIG. 11B), a sound generation request, and a light emission request at the determined timing is waited for (Step 104 NO, Step 107 NO, and Step 109 NO). Here, the spraying request is to be internally generated, and when there is a request, it is “1”, while when there is no request, it is “0” as illustrated in FIG. 11B.

If the spray request is generated in this state (Step 104 YES), then, after detected voltage determining processing (details will be described later) is executed (Step 105), by referring to a determination result of the detected voltage Vx, determination is made on whether the contents is “1” or “0” (Step 106). Here, if the determination result of the detected voltage Vx is “1” (there is water droplet) (Step 106 “1”), the ON state of the sprayer driving signal S1 and the light emission signal S3 are output from the output port PO1, PO3 (Steps 107, 108). On the other hand, if the determination result of the detected voltage Vx is “0” (no water droplet) (Step 106 “0”), the outputs of the ON state of the aforementioned sprayer driving signal S1 (Step 107) and the light emission signal (Step 108) are skipped as a protective operation, and instead, the OFF state of the sprayer driving signal S1 is output (Step 109). As described above, when the sprayer driving signal S1 indicates the ON state, the driving signal enters the oscillating state, and the mist generating operation is performed, while when the sprayer driving signal S1 indicates OFF state, the driving circuit enters the oscillation stopped state, and the mist generating operation is not performed. As a result, metal fatigue accumulation caused by the idle vibration of the piezoelectric vibrating plate 114 constituting the vibrator is avoided. Moreover, as described above, when the light emission signal S3 is output (Step 109 YES), the mist passing through the funnel 102 is illuminated in red, for example, by lighting or flashing the light emission diode 115, and the effect as if the light leaks from the combustion furnace is performed. In FIGS. 11A-11D, the waveform of the vehicle-speed pulse (in the FIG. 11D) is only a reference, and it should be noted that timing relationships with its cycle and other waveforms are not necessarily accurate.

If the determination result of the detected voltage Vx is “0” (no water droplet) (Step 106 NO), as the protective operation, notification of the water shortage state or prompting of water supply may be made by lighting an alarm lamp provided separately, by displaying alarm characters on a display provided separately or emitting an alarm sound through the speaker 126 instead of or together with prohibition of the mist generating operation.

If the sound emission request is generated during the processing above (Step 110 YES), output processing of the sound emission signal S2 from the output port PO2 (Step 111) is executed. Here, as described above, the sound emission signal S2 is an audio signal for driving the speaker 126, and its contents may be a Russel sound emitted by the steam locomotive or a voice talking to children (“I am . . . ”, “Now, passing by . . . ”, for example).

2) Detected Voltage Determining Processing (Step 105)

A flowchart schematically illustrating an example of the detected voltage determining processing is illustrated in FIG. 16. In this figure, when the processing is started, the internal switch SW2 incorporated in the CPU 127 is switched from the GND side to the Vcc side (Step 201), and a timer specifying the maximum waiting time Tw (100 μsec, for example) is started at the same time (Step 202) and then, during a period of time until the timer is timed up (Step 205 NO), the reading processing (Step 203) of the detected voltage Vx from the input port PI2 and the comparison processing (Step 204) with the threshold value voltage Vth are repeatedly executed. During that time, if the detected voltage Vx exceeds the threshold value voltage Vth (Step 204 YES), the determination result of the detected voltage is stored as “0” (no water) (Step 206). On the other hand, if the detected voltage Vx does not exceed the threshold value voltage Vth and the timer is not timed up (Step 205 YES), the determination result of the detected voltage Vx is stored as “1” (there is water) (Step 206). When either one of two determination result storage processing (Steps 206, 207) is completed, the internal switch SW2 is immediately switched from the Vcc side to the GND side (Step 208) and then, the processing is finished. Thus, whether the metal thin plate of the piezoelectric vibrating plate 114 constituting the vibrator is in contact with water or not can be confirmed on the basis of whether the result of the detected voltage determining processing is “1” or “0”.

### <Another Embodiment for Liquid-Contact Detection>

1) Detection Circuit

Subsequently, another example of the detection circuit will be described. In this example, since presence of liquid-contact with the vibrating plate is detected on the basis of the static capacitance value not on the basis of the electric resistance value between the first detecting electrode and the second detecting electrode, it has a merit that it is particularly effective as a measure against electric corrosion of the detecting electrode.

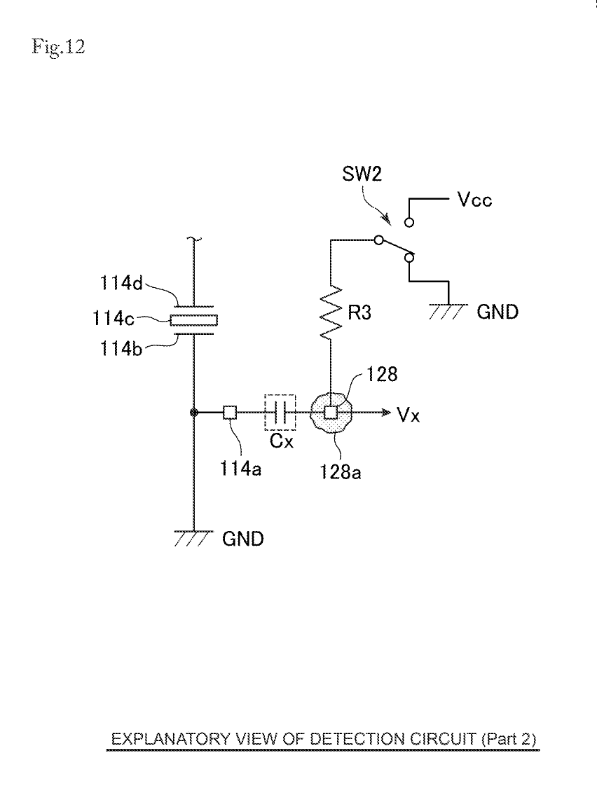

An explanatory view of the detection circuit (Part 2) is illustrated in FIG. 12. As illustrated in the figure, in this circuit, for at least either one of the first detecting electrode and the second detecting electrode (the second detecting electrode in this example), a projection-shaped electrode 128 having its periphery covered with a thin dielectric film 128a is employed. As the projection-shaped electrode 128 itself, corrosion resistance does not have to be considered since it is not in contact with water and thus, it may be constituted by a conductive metal of an arbitrary material. Since those other than the structure of the second detecting electrode are similar to the detection circuit described above by refereeing to FIG. 9, the explanation will be omitted.

The thinner a thickness of the dielectric film 128a is or the larger a relative dielectric constant of its material is, the more preferable it is for detection performances, but if a plastic resin is used as a material, a large dielectric constant cannot be expected and thus, a material which is thin and can hold mechanical strength to some degree is selected. When coating is employed as a film forming method, Teflon (registered trademark), epoxy, polyester and the like can be cited as the material. When putting a cap is employed as the film forming method, PVC, silicon or the like can be cited as a material of the cap. Moreover, by using aluminum as a conductive electrode to be a core and by applying alumite processing to its surface, the second detecting electrode with an extremely thin film having a high dielectric constant can be realized.

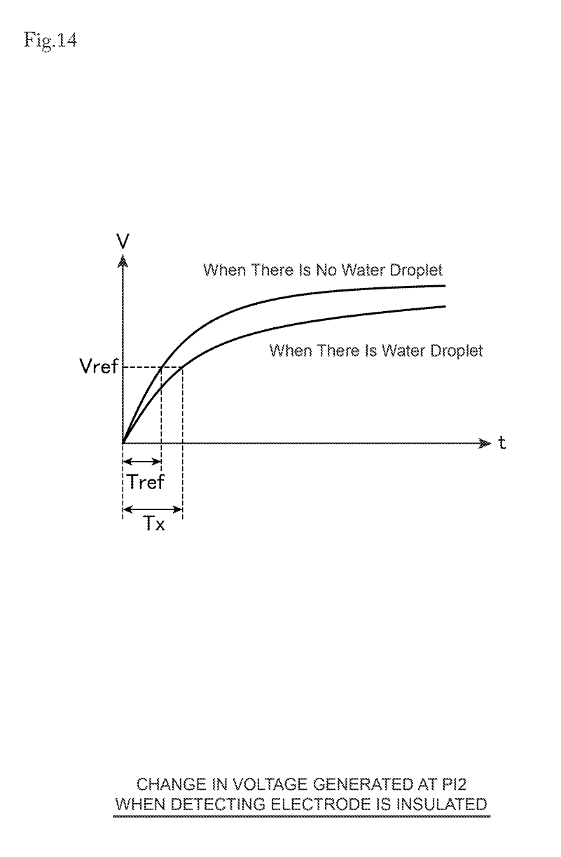

Subsequently, a detection principle of the water using the aforementioned detection circuit will be described. A graph showing a change in the voltage generated at the input port PI2 when either one of the detecting electrodes is insulated by the dielectric film is illustrating in FIG. 14. This graph shows a voltage change at the point of time when the internal switch SW2 is switched from the GND side to the Vcc side and after. In FIG. 14, an upper curve is a charging curve of the static capacitance Cx between the both electrodes in a state (first state) where there is no water between the first detecting electrode (metal thin plate 114a) and the second detecting electrode (projection-shaped electrode 128 with dielectric film), while a lower curve is the charging curve of the static capacitance Cx between the both electrodes in a state (second state) where there is water between the first detecting electrode and the second detecting electrode. As is obvious from the figure, inclination of a rising portion of the charging curve is gentler in the second state with water than in the first state without water between the both electrodes. Therefore, by comparing charging time Tx from charging start to a certain reference voltage Vref in the charging curve at an arbitrary static capacitance with charging time Tref from the charging start in the first state without water to the reference voltage Vref, presence of contact of the water with the vibrating plate can be detected. A value of Tref is generated by an influence of stray capacitance of the wiring to the second detecting electrode 128, and it is only necessary to measure it in advance as an initial value in the state without water droplet and to hold it as a known value. If a surface area of the second detecting electrode 128 is small, a value of Tx−Tref becomes an extremely small value. In such a case, by comparing/determining integrated values repeatedly measured several to several tens of times, more reliable determination result can be obtained. The larger the value of the pull resistance R3 is, the larger the values of Tref and Tx become, which rises resolution in numerical value calculation in the CPU and contributes to improvement of detection sensitivity by improved measurement accuracy, but considering an influence of a disturbance noise, approximately several 10 k to several 100 kΩ is preferable.

2) Detected Voltage Determination Processing

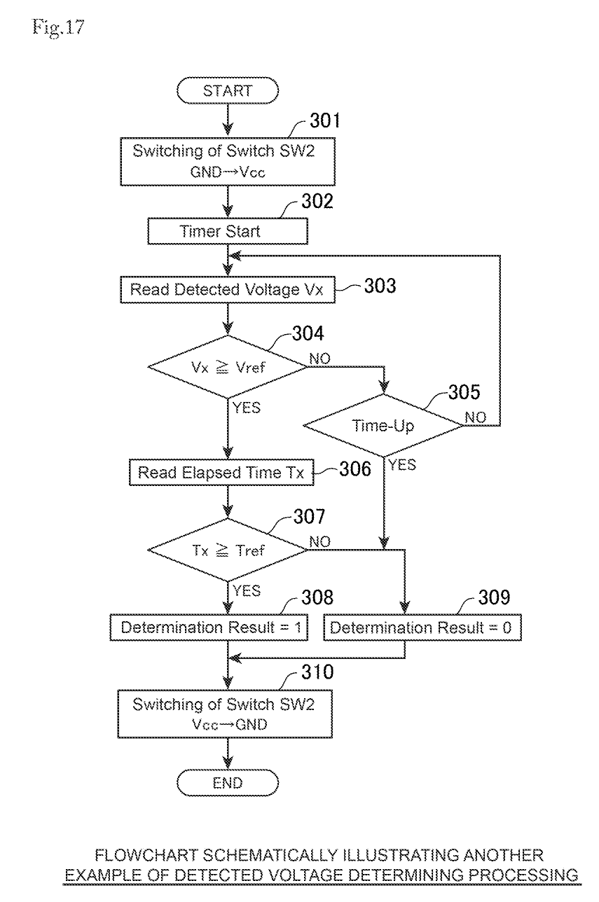

A flowchart schematically illustrating an example of the detected voltage determination processing using the aforementioned detection circuit (see FIG. 12) is illustrated in FIG. 17. In this figure, when the processing is started, first, by switching the internal switch SW2 from the GND side to the Vcc side, the potential at the output port PO4 is raised from the GND potential to the Vcc potential (Step 302). Subsequently, after the timer for clocking is started (Step 302), the reading processing of the detected voltage Vx (Step 303) and comparison processing between the detected voltage Vx and the reference voltage Vref (Step 304) are repeatedly executed until the timer is timed-up (Step 305 NO). During that time, if it is determined that the detected voltage Vx exceeds the reference voltage Vref (Step 304 YES), clocking time Tx of the timer is read (Step 306) and then, the clocked time Tx and the reference time Tref are compared with each other (Step 307). Here, if it is determined that the value of the clocked time Tx is larger than the value of the reference time Tref (Step 307 YES), the determination result of the detected voltage Vx is stored as “1” (there is water). On the other hand, if it is determined that the value of the clocked time Tx is less than the value of the reference time Tref or if it is determined that the timer is timed-up before the value of the clocked time Tx before the value of the detected voltage Vx reaches the reference voltage Vref (Step 305 YES), the determination result of the detected voltage Vx is stored as “0” (no water). Subsequently, the internal switch SW2 is switched from the Vcc side to the GND side, the potential of the output port PO4 is raised from the Vcc potential to the GND potential, and the processing is finished (Step 310). Thus, whether the metal thin plate of the piezoelectric vibrating plate 114 constituting the vibrator is in contact with the water or not can be confirmed on the basis of the result showing whether the detected voltage determination processing is “1” or “0”.

### <Notification of Water-Supply Completion>

In the aforementioned embodiment, whether the metal thin plate of the piezoelectric vibrating plate 114 constituting the vibrator is in contact with the water or not can be confirmed by the detection circuit illustrated in FIG. 9 or 12 and the detected voltage determination processing illustrated in FIG. 16 or 17. Thus, this function can be also used for notification of the water-supply completion. In that case, it may be configured such that, at the initialization processing (Step 101) in the flowchart illustrated in FIG. 15, for example, transfer to the routine processing (Steps 102 to 111) is waited for while the detected voltage determination processing (Step 105) is repeatedly executed, and when a change of the detected voltage determination result from “0” (no water) to “1” (there is water), the ON state of the sprayer driving signal S1 is output, and the spraying operation is performed in the predetermined mode so that the smoke is blown out from the funnel 102. It may be naturally configured such that the water-supply completion is notified by light and sound by outputting the light emission signal S3 and/or the sound emission signal S2 together with the spraying driving signal S1.

### <Others>

In the aforementioned embodiments, the vibrating plate is not limited to the piezoelectric vibrating plate 114 having the aforementioned specific structure, but vibrating plates with various conventional structures such as a vibrator itself constituted by sandwiching a piezoelectric material between a pair of driving electrodes (see Japanese Utility Model Registration Laid-Open No. 05-070592) or a metal tongue cantilever-supported by the aforementioned vibrator (see Japanese Patent Laid-Open No. 04-150968 and Japanese Patent No. 3744931) can be employed as the vibrating plate.

Moreover, the liquid supply mechanism is not limited to the aforementioned inclined gutter 113, but various conventional structures such as a mechanism for generating a mist by dripping a liquid stored in a liquid storage tank onto the vibrating plate in a horizontal posture through a tube with a flow regulating valve (Japanese Patent Laid-Open No. 04-150968 and Japanese Utility Model Registration Laid-Open No. 05-070592) or a mechanism for generating a mist from an upper surface side by supplying a liquid to a lower surface of the vibrating plate through a liquid retaining material such as a sponge placed on the lower surface of the vibrating plate with fine holes and having a substantially horizontal posture (Japanese Patent No. 3744931) and the like can be employed as the liquid supply mechanism.

Moreover, the mist generating device according to the present disclosure can be widely employed in various toys performing effects of smoke (an automobile toy blowing out smoke from an exhaust pipe, a water fountain toy blowing out water smoke and the like) other than the steam locomotive toy.

According to the present disclosure, when such a state emerges where the liquid which is the mist material is not brought into contact with the vibrating plate due to various reasons caused by the structure of the liquid supply mechanism such that the liquid storage tank is emptied, a liquid supply path from the liquid storage tank to the vibrating plate is clogged, a sponge which is a liquid retaining material is dried or the like, the protective operation for preventing fatigue accumulation caused by idle vibration of the vibrating plate is immediately performed and as a result, defective generation or incapable generation of mist due to aging degradation or breakage of the vibrating plate can be prevented.

Moreover, since presence of liquid-contact with the vibrating plate itself located at the end of the liquid supply path is detected instead of a liquid level of the liquid storage tank located in the middle of the liquid supply path or electrical conductivity of the liquid retaining material (sponge, for example), by means of the innovative design such that the liquid storage tank or the liquid retaining material is removed and a liquid amount required for one mist generation cycle (several tens of seconds, for example) is supplied to the vibrating plate each time, situations such as fungi growth, generation of odor, deposition of calcium carbonate or the like due to leaving of the used liquid for a long time in the liquid storage tank or the liquid retaining material can be also prevented.

### REFERENCE SIGNS LIST

1 steam locomotive toy
2 freight vehicle toy
3 track
3a guiding projection
3b guiding projection
4 iron bridge
5 water storage tank
5a operation button
5b water-supply nozzle
6 recess portion (pond)
7 operation lever
101 outer shell
102 funnel
103 water inlet
104 slide operator (power switch)
105 sound emission hole
106 water outlet
107a front left wheel
107b front right wheel
108a rear left wheel
108b rear right wheel
109a axle of front wheels
109b axle of rear wheels
110a rear-end mounting hole of left-side rod
110b rear-end mounting hole of right-side rod
111a front-rear wheel connecting rod on left side
111b front-rear wheel connecting rod on right side
112a front-end mounting long hole of left-side rod
112b front-end mounting long hole of right-side rod
113 inclined gutter
113a floor surface of inclined gutter
113b upstream portion of inclined gutter
113c downstream end wall of inclined gutter
114 piezoelectric vibrating plate
114a disc-shaped metal thin plate (first detecting electrode)
114b annular first driving electrode
114c annular piezoelectric material layer
114d annular second driving electrode
114e insulating film
115 light emission diode (LED)
116 swing lever
117 base end portion
118 operator
119 detection switch
120 cam
121 gap
122 projection-shaped electrode (second detecting electrode)
123 small circular region
124 water droplet
125 mist
126 speaker
127 CPU
128 projection-shaped electrode with dielectric film
128a dielectric film
201 switch
R1 pull resistance
R2 resistance for current return
R3 pull resistance
Rx inter-electrode resistance
Cx inter-electrode static capacitance
Vx detected voltage
E power supply

What is claimed is:

1. A mist generating device comprising a vibrating plate which vibrates at a high frequency and a liquid supply mechanism for supplying a conductive liquid such as water to the vibrating plate and generating a mist by bringing the liquid supplied through the liquid supply mechanism into contact with the vibrating plate for atomization, the mist generating device further comprising:
liquid-contact detecting unit for detecting presence of contact of the liquid with the vibrating plate; and
protective operation performing unit for performing a protective operation for preventing idle vibration of the vibrating plate when the liquid-contact detecting unit detects non-contact of the liquid with the vibrating plate.

2. The mist generating device according to claim 1, wherein
the protective operation is an operation for prohibiting vibration of the vibrating plate.

3. The mist generating device according to claim 1, wherein
the liquid-contact detecting unit includes:
first and second detecting electrodes between which the liquid is filled only during a period when the liquid is in contact with the vibrating plate; and
determining unit for determining presence of contact of the liquid with the vibrating plate on the basis of impedance between the first detecting electrode and the second detecting electrode.

4. The mist generating device according to claim 3, wherein
by giving the same potential to both ends of a series circuit of the impedance between the both electrodes and a pull resistance at non-detection, while giving a known potential difference to the both ends of the series circuit at detection, the impedance is detected through a voltage drop generated in the pull resistance at that time.

5. The mist generating device according to claim 3, wherein
the first detecting electrode and the second detecting electrode are both made of bare conductors, and the impedance is electric resistance.

6. The mist generating device according to claim 3, wherein
at least either one of the first detecting electrode and the second detecting electrode is made of a conductor having a thin dielectric film on a surface, and the impedance is capacitive reactance.

7. The mist generating device according to claim 1, wherein
the liquid supply mechanism has an injected liquid guiding portion for guiding a slight amount of the liquid injected or dripped through an inlet to the vibrating plate and a slight-amount liquid holding portion for holding the slight amount of liquid guided by the injected liquid guiding portion in a state in contact with the vibrating plate until it is completely consumed by a mist generating action.

8. The mist generating device according to claim 7, wherein
the slight-amount liquid holding portion holds the liquid in the state in contact with the vibrating plate by using a surface tension of the liquid.

9. The mist generating device according to claim 7, wherein
the vibrating plate is a vibrating plate with fine holes in which either one of front and rear surfaces is a liquid contact surface, while the other surface is a mist emission surface and arranged in a posture with the mist emission surface directed upward;
the injected liquid guiding portion is an inclined gutter arranged so that its upstream end is located at a liquid inlet, while a downstream end is located on a lower side of the vibrating plate with fine holes; and
the slight-amount liquid holding portion is a narrow gap formed between the lower surface of the vibrating plate with fine holes and an upper surface of a gutter floor of the inclined gutter.

10. The mist generating device according to claim 9, wherein
the vibrating plate with fine holes is a piezoelectric vibrating plate formed by sequentially laminating and integrating a metal thin plate having fine holes, an annular first driving electrode, an annular piezoelectric material layer, and an annular second driving electrode and by insulating/covering a periphery of the metal thin plate while leaving the front and the rear; and
the first detecting electrode is the metal thin plate, and the second detecting electrode is a projection-shaped electrode provided on a floor surface of the gutter.

11. A mist generating device having a vibrating plate which vibrates at a high frequency and a liquid supply mechanism for supplying a conductive liquid such as water to the vibrating plate and generating a mist by bringing the liquid supplied through the liquid supply mechanism into contact with the vibrating plate for atomization, the mist generating device further comprising:
liquid-contact detecting unit for detecting presence of contact of the liquid with the vibrating plate; and
notification operation performing unit for performing a notification operation for notifying completion of the liquid supply when the liquid-contact detecting unit detects a change from non-contact to contact of the liquid with the vibrating plate.

12. The mist generating device according to claim 11, wherein
the notification operation is an operation for notifying completion of the liquid supply operation through generation of a mist by vibrating the vibrating plate in a predetermined mode.

13. The mist generating device according to claim 1, wherein
the mist generating device is incorporated in a toy performing an effect of smoke or water smoke.

14. A steam locomotive toy in which a mist generating device having a vibrating plate which vibrates at a high frequency and a liquid supply mechanism for supplying a conductive liquid such as water to the vibrating plate and generating a mist by bringing the liquid supplied through the liquid supply mechanism into contact with the vibrating plate for atomization is incorporated inside an outer shell copying an appearance of a steam locomotive and performing an effect of smoke by discharging the mist generated in the mist generating device to an outside through a funnel provided on the outer shell, wherein
the liquid supply mechanism has:
an injected liquid guiding portion for guiding a slight amount of liquid injected or dripped through an inlet provided in the outer shell to the vibrating plate; and
a slight-amount liquid holding portion for holding the slight amount of the liquid guided by the injected liquid guiding portion in a state in contact with the vibrating plate until it is completely consumed by a mist generating action.

15. The steam locomotive toy according to claim 14, wherein
the slight-amount liquid holding portion holds the liquid in a state in contact with the vibrating plate by using the surface tension of the liquid.

16. The steam locomotive toy according to claim 14, wherein
the vibrating plate is a vibrating plate with fine holes in which either one of front and rear surfaces is a liquid contact surface, while the other surface is a mist emission surface and arranged in a posture with the mist emission surface directed upward;
the injected liquid guiding portion is an inclined gutter arranged so that its upstream end is located at a liquid inlet, while a downstream end is located on a lower side of the vibrating plate with fine holes; and
the slight-amount liquid holding portion is a narrow gap formed between the lower surface of the vibrating plate with fine holes and an upper surface of a gutter floor of the inclined gutter.

17. A steam locomotive toy system comprising:
a track;
a railway station provided in the middle of the track; and
the steam locomotive toy according to claim 14, wherein
at the railway station, a liquid injection facility having a liquid injection nozzle for injecting a slight amount of the liquid into a liquid inlet of the steam locomotive toy stopped at the railway station by a predetermined liquid injecting operation is provided.

18. The steam locomotive toy system according to claim 17, wherein
in the outer shell of the steam locomotive toy, an outlet for discharging the liquid overflowing from the vibrating plate is provided; and
at the railway station on the track, a recess portion for storing the liquid flowing out of the outlet of the stopped steam locomotive toy is provided.
